(19) United States
(12) Patent Application Publication
(10) Pub. No.: US 2025/0001071 A1
(43) Pub. Date: Jan. 2, 2025

(54) INFUSION PUMP

(71) Applicant: Takeda Pharmaceutical Company Limited, Osaka-shi, Osaka (JP)

(72) Inventors: Jack Lawrence Warren, Royston (GB); Alexander David Norman, Cambridge (GB); Merissa Lim Sarrias, Hauxton (GB)

(73) Assignee: Takeda Pharmaceutical Company Limited, Osaka-shi, Osaka (JP)

(21) Appl. No.: 18/689,723

(22) PCT Filed: Sep. 9, 2022
(86) PCT No.: PCT/IB2022/000507

§ 371 Date: Mar. 6, 2024

Related U.S. Application Data
(60) Provisional application No. 63/242,968, filed on Sep. 10, 2021.
(60) Provisional application No. 63/396,926, filed on Aug. 10, 2022.
(60) Provisional application No. 63/397,350, filed on Aug. 11, 2022.

(51) Int. Cl. A61M 5/145; A61M 5/178; A61M 5/315

(52) U.S. Cl. CPC A61M 5/1456; A61M 5/1782; A61M 5/31581

(57) ABSTRACT

An infusion pump may include automated syringe loading capabilities and/or medicinal fluid delivery capabilities to facilitate administration of medicinal fluids to a patient. The infusion pump may operate mechanically and may not require a power source to operate. The infusion pump may assist with filling a syringe with the medicinal fluid contents of one or more vials. The infusion pump may include a drug delivery assembly to deliver fluid at an adjustable but constant force. The infusion pump may permit the user to adjust the delivery rate continuously within a given range to improve comfort during infusion.

### CROSS-REFERENCE TO RELATED APPLICATIONS

This application claims the benefit under 35 U.S.C. 119(e) of U.S. Provisional Application No. 63/242,968 filed on Sep. 10, 2021, U.S. Provisional Application No. 63/396,926 filed on Aug. 10, 2022, and U.S. Provisional Application No. 63/397,350 filed on Aug. 11, 2022, the entireties of which are incorporated herein by reference.

### FIELD

Disclosed embodiments are related to infusion pumps and related methods of use.

### BACKGROUND

Medicinal fluids are administered to patients through a variety of methods. These conventional methods typically include injection by a syringe, ingestion, or delivery by an infusion pump and needle. In the case of administration by an infusion pump, controlled volumes of medicinal fluids may be delivered to the patient at pre-programmed rates or automated intervals. Conventional infusion pumps can be expensive, complex to operate, and can require delicate cleaning processes performed by a health care provider.

Typically, the delivery of medicinal fluids by an infusion pump is performed by an experienced health care provider who is responsible for operating the infusion pump to administer medicinal fluid as well as any maintenance or cleaning the infusion pump might require. A health care provider will also connect any accessories to the infusion pump to facilitate infusion, including needlesets, tubing, and containers of medicinal fluid.

### SUMMARY

In some embodiments, an infusion pump includes a holder configured to push and pull a syringe plunger, the holder configured to couple to the syringe plunger, the holder being moveable between a first position and a second position, and the holder being biased towards the first position, a loading actuator operatively coupled to the holder, wherein operation of the loading actuator causes the holder to move from the first position to the second position, and a lock configured to releasably fix the holder in the second position.

In some embodiments, a method of operating an infusion pump includes providing an infusion pump having a holder and a port, coupling a syringe plunger of a syringe to the holder, fluidly coupling a vial to the port, and moving the holder from a first position to a second position to cause the holder to move the syringe plunger in a fill direction and to cause liquid medicament from the vial to flow through the port and enter the syringe.

In some embodiments, an infusion pump includes a holder configured to removably couple to a syringe plunger, a loading actuator operatively coupled to the holder, the loading actuator being moveable between a first position and a second position, an energy storing member operatively coupled between the holder and the loading actuator, wherein movement of the loading actuator from the first position to the second position loads the energy storing member, and a lock configured to releasably fix the loading actuator in the second position.

In some embodiments, a method of operating an infusion pump includes providing an infusion pump having a holder, coupling a syringe plunger of a syringe to the holder, moving a loading actuator from a first position to a second position, causing loading of an energy storing member that is operatively coupled between the holder and the loading actuator, and locking the loading actuator in the second position. In some embodiments, the loaded energy storing member exerts a force on the holder, causing the holder to move the syringe plunger in a fill direction, and causing liquid medicament from a vial to enter the syringe.

In some embodiments, an infusion pump includes a contact surface configured to contact a syringe plunger, the contact surface being moveable between a first position and a second position, an energy storing assembly configured to move the contact surface from the second position to the first position by applying a substantially constant force to the contact surface, and a rate actuator operatively coupled to the energy storing assembly, the rate actuator configured to mechanically adjust a magnitude of the substantially constant force.

In some embodiments, a method of operating an infusion pump includes operating a loading actuator to load an energy storing assembly, the energy storing assembly becoming locked in a loaded state, unlocking the energy storing assembly from the loaded state, causing the energy storing assembly to unload and to produce a substantially constant force that is transmitted to a contact surface, causing the contact surface to abut against and move a syringe plunger in a dispensing direction, and operating a rate actuator to mechanically adjust a magnitude of the substantially constant force.

In some embodiments, an infusion pump includes a contact surface configured to contact a syringe plunger, the contact surface being moveable between a first position and a second position, and an energy storing assembly configured to move the contact surface from the second position to the first position by applying a substantially constant force to the contact surface. In some embodiments, the energy storing assembly includes a linkage operatively coupled to the contact surface, and an energy storing member. In some embodiments, the linkage is configured to convert a variable output force of the energy storing member into the substantially constant force.

In some embodiments, an infusion pump may include a first sledge operable by a loading actuator. The first sledge may be configured to linearly displace a syringe plunger. The infusion pump may include a second sledge that is operatively coupled to the first sledge. The infusion pump may include a lock engageable with the second sledge to retain the second sledge in a locked configuration. In some embodiments, the lock may be configured to permit the second sledge to be driven by energy stored within an energy storing member to linearly displace the first sledge in a dispensing direction in an unlocked configuration.

In some embodiments, a method of operating an infusion pump may include operating a loading actuator to linearly displace a first sledge, the first sledge configured to linearly displace a syringe plunger. The method may also include retaining a second sledge in a locked configuration with a lock engageable with the second sledge, wherein the second sledge is operatively coupled to the first sledge. The method may also include unlocking the lock to permit the second sledge to be driven by energy stored within an energy storing member to linearly displace the first sledge in a dispensing direction in an unlocked configuration.

In some embodiments, an infusion pump may include a holder configured to removably couple to a syringe and to push and pull at least a portion of the syringe. The infusion pump may include a loading actuator that is operatively coupled to the holder. The loading actuator may be configured to displace the holder. The infusion pump may include a port in removable fluid communication with the syringe, the port configured to removably couple to a fluid container. Operation of the loading actuator may urge fluid flow from the fluid container to the syringe.

In some embodiments, a method of operating an infusion pump may include operating a loading actuator that is operatively coupled to a holder to displace the holder. The holder may be configured to removably couple to a syringe and to push and pull at least a portion of the syringe. The method may include displacing the holder with the loading actuator. The method may include removably coupling a fluid container to a port. The port may be in removable fluid communication with the syringe. Operation of the loading actuator may urge fluid flow from the fluid container to the syringe.

In some embodiments, an infusion pump may include a holder configured to removably couple to a syringe plunger and to push and pull the syringe plunger. The infusion pump may include a rate actuator that is operatively coupled to the holder. The infusion pump may include an energy storing member that is operatively coupled between the rate actuator and the holder. In some embodiments, operation of the rate actuator may mechanically adjust energy stored within the energy storing member. The infusion pump may include a lock engageable with the holder to permit force transfer between the energy storing member and the holder to displace the holder when the lock is in an unlocked configuration.

In some embodiments, a method of operating an infusion pump may include operating a rate actuator to mechanically adjust energy stored within an energy storing member. The energy storing member may be operatively coupled between the rate actuator and a holder. The holder may be configured to removably couple to a syringe plunger and to push and pull the syringe plunger. The method may include permitting force transfer between the energy storing member and the holder by arranging a lock in an unlocked configuration. The lock may be engageable with the holder.

In some embodiments, an infusion pump may include a locking actuator configured to permit fluid flow out of a syringe in an unlocked configuration. The locking actuator may be configured to block fluid flow out of the syringe in a locked configuration. The infusion pump may include a ratchet wheel configured to engage with a portion of the locking actuator in the locked configuration of the locking actuator. The ratchet wheel may be rotationally coupled to a first gear. The infusion pump may include a second gear configured to engage with the first gear, the second gear being operatively coupled to the syringe. A gear ratio between the second gear and the first gear may be greater than 1:1.

In some embodiments, a method of operating an infusion pump may include permitting fluid flow out of a syringe by arranging a locking actuator in an unlocked configuration. The method may include blocking fluid flow out of the syringe by arranging the locking actuator in a locked configuration to engage a ratchet wheel with a portion of the locking actuator. The ratchet wheel may be rotationally coupled to a first gear. The first gear may be configured to engage with a second gear that is operatively coupled to the syringe. A gear ratio between the second gear and the first gear may be greater than 1:1.

In some embodiments, an infusion pump may include a holder configured to removably couple to a syringe plunger and to push and pull the syringe plunger. The infusion pump may include a loading actuator that is operatively coupled to the holder. Operation of the loading actuator may cause the holder to push and pull the syringe plunger. The infusion pump may include a lock configured to permit fluid flow out of a syringe barrel associated with the syringe plunger in an unlocked configuration. The lock may be configured to block fluid flow out of the syringe barrel in a locked configuration. The lock may be configured to releasably de-couple the holder and the loading actuator in the locked configuration.

In some embodiments, a method of operating an infusion pump may include operating a loading actuator to linearly actuate a holder. The holder may be configured to removably couple to a syringe plunger. The holder may be configured to push and pull the syringe plunger, permitting fluid flow out of a syringe barrel associated with the syringe plunger by arranging a lock in an unlocked configuration. The method may include blocking fluid flow out of the syringe barrel by arranging the lock in a locked configuration. The method may include releasably de-coupling the holder and the loading actuator in the locked configuration.

In some embodiments, an infusion pump may include a holder configured to removably couple to a syringe plunger and to push and pull the syringe plunger. The infusion pump may include an energy storing assembly configured to apply a substantially constant force to the holder to displace the holder. In some embodiments, the energy storing assembly may include an energy storing member configured to provide a variable output force. The energy storing assembly may include a drive arm configured to apply the substantially constant force to the holder. The infusion pump may include a linkage that is operatively coupled between the drive arm and the energy storing member. The linkage may be configured to convert the variable output force of the energy storing member into the substantially constant force.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1C depict schematics of a process of operating an infusion pump according to some embodiments;

FIG. 2 is a flow chart of an embodiment of a method for operating the infusion pump of FIGS. 1A-1C;

FIGS. 3A-3B depict schematics of a process of operating an infusion pump according to some embodiments;

FIG. 4 is a flow chart of an embodiment of a method for operating the infusion pump of FIGS. 3A-3B;

FIG. 5 is a schematic of another embodiment of an infusion pump;

FIG. 6 is a flow chart of an embodiment of a method for operating the infusion pump of FIG. 5;

FIG. 7 depicts a schematic of another embodiment of an infusion pump;

FIG. 8 is a perspective exploded view of a bubble trap according to some embodiments;

FIG. 9 is a front, perspective, top view of another embodiment of an infusion pump;

FIG. 10 is a rear, perspective, top view of the infusion pump of FIG. 9;

FIG. 11 is a front, perspective top view of the infusion pump of FIG. 9

FIG. 12 depicts a schematic of yet another embodiment of an infusion pump;

FIGS. 13A-13B are partial cross-sectional front, perspective, top views of the interior of the infusion pump of FIG. 12 during rate adjustment, according to some embodiments;

FIG. 14 is a back, perspective, top view of the interior of the infusion pump of FIG. 12

FIG. 15 is a front, perspective, top view of the interior of the infusion pump of FIG. 12;

FIG. 16 is a front, perspective, top view of a syringe latch, according to some embodiments;

FIG. 17 is a front, perspective, top view of an adaptor for use with the infusion pump of FIG. 12, according to some embodiments;

FIGS. 18A-18B are front, perspective, top views of the interior of the infusion pump of FIG. 12 in the process of being unlocked, according to some embodiments;

FIGS. 19A-19B are top views of the interior of the infusion pump of FIG. 12 in the process of being unlocked, according to some embodiments;

FIGS. 20A-20B are partial front, perspective, top view of the clutch mechanism of the infusion pump of FIG. 12 according to some embodiments;

FIGS. 21A-21B are partial front, perspective, top views of the interior of the infusion pump of FIG. 12 in the process of being unlocked, according to some embodiments;

FIG. 22 is a flow chart of an embodiment of a method for loading the infusion pump of FIG. 12;

FIG. 23 is a flow chart of an embodiment of a method for infusing with the infusion pump of FIG. 12; and

FIG. 24 is a flow chart of an embodiment of a method for unloading the infusion pump of FIG. 12.

### DETAILED DESCRIPTION

During a typical administration process of medicinal fluid using an infusion pump, a nurse or other health care provider performs numerous steps to prepare the infusion pump for operation, operates the pump to deliver the medicinal fluid, and maintains the pump for subsequent administrations. At each step, the health care provider takes care to maintain sterility as fittings, medicinal fluid containers, and other accessories are connected and disconnected from the infusion pump. Generally, the health care provider undertakes regularly scheduled cleanings of an infusion pump which are complex processes which require numerous steps to complete. Accordingly, conventional infusion pumps are expensive, difficult to operate, and typically require time consuming steps to set up and operate.

In some cases, due to the type, duration and/or frequency of treatment using some medicinal fluids, self-administration can be a desirable option for convenience and/or cost. Infusion pumps which are already complex and difficult to operate for health care providers can be even more challenging to operate and maintain for a patient practicing self-administration. For example, a patient may need to fill one or more syringes with medicinal fluid from one or more vials, connect new needlesets for each treatment, align and connect tubing to the pump, handle and connect one or more containers of medicinal fluid, and perform other steps for a single administration which may be difficult and time consuming. Additionally, a patient may need to perform complicated cleaning and maintenance processes to prepare their infusion pump for subsequent administrations.

The inventors have recognized a need for an infusion pump with reduced complexity, inexpensive operation (e.g., not requiring costly and/or specialized equipment or processes), and improved sterility of medicinal fluid administration which may be readily used by health care professionals and/or self-administering patients (hereinafter referred to as “users”).

In view of the above, the inventors have recognized the benefits of an inexpensive and mechanically operated infusion pump that may be used to deliver a medicinal fluid in an easy to operate manner. The infusion pump may be configured to subcutaneously deliver a large volume of medicinal fluid at a desired fluid flow rate.

According to exemplary embodiments described herein, an infusion pump may be used with any number of medicinal or nutritional fluids (both of which are hereinafter referred to as “fluid”) which are delivered to the body (e.g., subcutaneously). In some embodiments, the fluid may be a liquid medicament. In some embodiments, an infusion pump may be configured to deliver Immune Globulin Infusion 10% (Human), Immune Globulin Subcutaneous (Human) 20% (e.g., CUVITRU), Recombinant Human Hyaluronidase (e.g., HYQVIA), other immune globulin drugs, and/or any other suitable medicament. Infusion pumps described herein may, in some embodiments, be configured to deliver fluids having a viscosity between 10 and 30 cP at 35° C., between 1 and 4.54 cP at 35° C. or any other suitable viscosity.

With conventional syringe infusion pumps, a syringe is typically manually filled by a user with fluid from one or more vials prior to coupling the syringe to the infusion pump. With such arrangements, the infusion pump itself does not assist the user in filling the syringe. The process of filling the syringe can be cumbersome and time-consuming. Thus, the inventors have recognized the need for an infusion pump that assists users with loading fluid into a syringe. In some embodiments, the infusion pump may include a syringe loading assembly that helps to reduce the amount of force and/or coordination needed from the user. In some embodiments, a syringe loading assembly may be configured to reduce air bubbles during loading of the syringe with fluid.

In some embodiments, the syringe loading assembly of an infusion pump may be purely mechanically operated. In other words, the process of loading a syringe with fluid using the infusion pump may occur mechanically, without requiring electricity. In some embodiments, the syringe loading assembly may include a user-operable handle to allow a user to select the volume of fluid to be loaded into a syringe. The handle may be operatively coupled to a plunger of the syringe, such that movement of the handle may translate the plunger through a barrel of the syringe. In some embodiments, the handle may be operatively coupled to a plunger via an elastic coupling. In some embodiments, the force applied to the handle by the user may be modulated by the elastic coupling, and the modulated force may then be transferred to the plunger by the energy storing member. In some embodiments, the elastic coupling may include an energy storing member. In some embodiments, the energy storing member may allow the plunger to translate at a lower speed than the speed at which the handle was moved. In some embodiments, the slower translation of the plunger may result in less frothing in the syringe. As a result, the filling of the syringe may not need to rely on a user spending time pulling back on the syringe plunger sufficiently slowly (e.g., to avoid potential bubble formation and/or damage to the medicament) and/or the user pulling back on the syringe plunger at a sufficiently steady rate. In some embodiments, the user may be able to move the handle to set a fill volume at any desired speed and rate without worrying about bubble formation and/or causing the syringe to fill too quickly.

In some embodiments, the syringe loading assembly of an infusion pump may be manually operated by mechanical means (e.g., without requiring electricity). In some embodiments, an infusion pump may include a user-operable loading actuator operatively coupled to a plunger holder configured to push or pull a syringe plunger relative to the syringe barrel. The user may operate the actuator to pull the plunger away from the barrel, forming a vacuum. As will be described in greater detail below, in cases where the syringe is in fluid communication with a vial of fluid, this movement may result in the syringe being filled with fluid. In some embodiments, the infusion pump may employ load reduction features such as gear trains to reduce the force and dexterity to operate the loading actuator.

In some embodiments, the infusion pump may be able to load a syringe by extracting fluid (e.g., medication) from any suitable pre-existing vial sizes and shapes. For example, the infusion pump may be able to extract fluid from a vial with a standard 20 mm opening, or any other suitable opening size. In some embodiments, the infusion pump may be able to extract fluid from vials containing at least 2 mL, 5 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, and/or any other suitable vial volume. In some embodiments, the infusion pump may be able to extract fluid from vials containing less than or equal to 500 mL, 450 mL, 400 mL, 350 mL, 300 mL, 250 mL, 200 mL, 150 mL, 100 mL, 95 mL, 90 mL, 85 mL, 80 mL, 75 mL, 70 mL, 65 mL, 60 mL, 55 mL, 50 mL, 45 mL, 40 mL, 35 mL, 30 mL, 25 mL, 20 mL, 15 mL, 10 mL, 5 mL, 2 mL, and/or any other suitable vial volume. Combinations of ranges for the vial volume are also contemplated, including between 5 mL and 50 mL, 2 mL and 500 mL, 10 mL and 30 mL, 50 mL and 500 mL, and/or any other suitable combinations of vial volumes. Of course, other ranges, including ranges both greater than and less than those noted above are also contemplated as the present disclosure is not so limited. It should be appreciated that the infusion pump may be able to extract fluid from any suitable vial volume, as the present disclosure is not so limited.

In some embodiments, the infusion pump may be able to sequentially extract fluid from one or more vials. In some embodiments, the infusion pump may be able to sequentially extract fluid from at least 1 vial, 2 vials, 3 vials, 4 vials, 5 vials, and/or any other suitable number of vials. In some embodiments, the infusion pump may be able to sequentially extract fluid from less than or equal to 5 vials, 4 vials, 3 vials, 2 vials, 1 vial, and/or any other suitable number of vials. Combinations of ranges for the number of vials used are also contemplated, including between 1 vial and 5 vials and between 2 vials and 5 vials, and/or any other suitable combination. Of course, other ranges, including ranges both greater than and less than those noted above are also contemplated as the present disclosure is not so limited. It should be appreciated that the infusion pump may be able to extract fluid from any number of vials, as the present disclosure is not so limited.

Existing infusion pumps with adjustable flow rates may include elastomeric components which may be incompatible with certain fluids. Thus, the inventors have also recognized the benefits of an infusion pump capable of delivering large volumes of fluid at an adjustable fluid delivery flow rate using components not directly in contact with the fluid. The infusion pump may have a user-operable control to allow the user to set the fluid delivery flow rate based on user input. The infusion pump may control the fluid delivery flow rate by controlling a force applied to a plunger of a syringe. The flow rate control may be integrated into the fluid delivery system, allowing a user to adjust the fluid delivery flow rate without throttling the fluid flow.

In some embodiments, an infusion pump may include a mechanically operated drug delivery assembly. The drug delivery assembly may include a variety of mechanical components arranged to apply an adjustable constant force to a plunger of a syringe. The constant force applied to the plunger may result in a substantially constant fluid flow rate out of the syringe. In some embodiments, the user may easily adjust the constant force by operating a rate actuator at any point (e.g., before, during, or after) of an infusion process. The user may adjust the fluid flow rate in response to comfort needs, timing needs (e.g., if the user has limited time to conduct the infusion process), biological needs (e.g., as recommended by a health care professional), or any other suitable set of needs. In some embodiments, the infusion pump may also include a lock or a toggle in order to stop and/or start infusion at any point during the infusion process.

In some embodiments, the infusion pump may deliver between 2 and 100 mL of a fluid during a single infusion process. In particular, an infusion pump may deliver a fluid volume of at least 2 mL, 5 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, and/or any other appropriate volume during a single infusion process. In some embodiments, the infusion pump may deliver a fluid volume less than or equal to 500 mL, 450 mL, 400 mL, 350 mL, 300 mL, 250 mL, 200 mL, 150 mL, 100 mL, 95 mL, 90 mL, 85 mL, 80 mL, 75 mL, 70 mL, 65 mL, 60 mL, 55 mL, 50 mL, 45 mL, 40 mL, 35 mL, 30 mL, 25 mL, 20 mL, 15 mL, 10 mL, 5 mL, 2 mL, and/or any other suitable volume. Combinations of ranges for the delivered fluid volume are also contemplated, including between 5 mL and 60 mL, 2 mL and 500 mL, 10 mL and 30 mL, 50 mL and 500 mL, and/or any other suitable combinations of delivered fluid volumes. Of course, other ranges, including ranges both greater than and less than those noted above are also contemplated as the present disclosure is not so limited. It should be appreciated that the infusion pump may be able to deliver any suitable volume of fluid, as the present disclosure is not so limited. In some embodiments, the infusion pump may deliver a substantial portion of the fluid loaded into the syringe (whether manually or mechanically, using the system described above). In some embodiments, the infusion pump may deliver greater than 95% of the fluid loaded into the syringe. Of course, embodiments in which the infusion pump may deliver significantly less fluid than is loaded in the syringe are also contemplated.

Additionally, the infusion pump may deliver fluids of the above-noted volumes with viscosities greater than 1 cP, 2.5 cP, 5 cP, 10 cP, 15 cP, 20 cP, 25 cP, 30 cP, 35 cP, and/or any other appropriate viscosity during a single infusion process. Correspondingly, the infusion pump may deliver fluids of the above-noted volumes with viscosities less than 50 cP, 45 cP, 40 cP, 30 cP, 20 cP, 15 cP, 10 cP, and/or any other appropriate viscosity during a single infusion process. In other embodiments, the infusion pump may deliver between 26.25 mL and 1260 mL of a fluid having a viscosity between 1 and 4.54 cP at 35° C. during a single infusion process. Combinations of ranges for the delivered fluid viscosities are also contemplated, including between 1 cP and 50 cP, 5 cP and 25 cP, and/or any other suitable combinations of delivered fluid viscosities. Of course, other ranges, including ranges both greater than and less than those noted above are also contemplated as the present disclosure is not so limited. It should be appreciated that the infusion pump may be able to deliver any suitable volume of fluid with any suitable viscosity, as the present disclosure is not so limited.

The user may be able to adjust the delivery flow rate of the fluid based on the properties of the fluid. In some embodiments, it may be desirable to infuse a more viscous fluid at a lower flow rate than a less viscous fluid. The delivery flow rate may be continuously adjustable in any desirable range. In other words, a user may be able to select any delivery flow rate within a given range, as opposed to step-wise, discontinuous flow rate settings. In some embodiments, the delivery flow rate may be continuously adjustable between 50 mL/hr and 300 mL/hr. As will be described in further detail below, the user may be able to adjust the delivery flow rate by manipulating a rate actuator. In some embodiments, the infusion pump may include visual markers on an external face of the pump to allow the user to select a desired flow rate.

In some embodiments, an infusion pump may deliver fluid at a rate of at least 10 mL/hr, 20 mL/hr, 30 mL/hr, 40 mL/hr, 50 mL/hr, 60 mL/hr, 80 mL/hr, 100 mL/hr, 120 mL/hr, 150 mL/hr, 180 mL/hr, 200 mL/hr, 250 mL/hr, 300 mL/hr, 400 mL/hr, 500 mL/hr, and/or any other suitable delivery flow rate. In some embodiments, the infusion pump may deliver fluid at a rate less than or equal to 500 mL/hr, 400 mL/hr, 300 mL/hr, 250 mL/hr, 200 mL/hr, 180 mL/hr, 150 mL/hr, 120 mL/hr, 100 mL/hr, 80 mL/hr, 60 mL/hr, 50 mL/hr, 40 mL/hr, 30 mL/hr, 20 mL/hr, 10 mL/hr, and/or any other suitable delivery flow rate. Combinations of ranges for the fluid delivery rate are also contemplated, including between 10 mL/hr and 500 mL/hr, 50 mL/hr and 300 mL/hr, and/or any other suitable combinations of fluid delivery flow rate. Of course, other ranges, including ranges both greater than and less than those noted above are also contemplated as the present disclosure is not so limited. It should be appreciated that the infusion pump may be able to deliver fluid at any suitable flow rate, as the present disclosure is not so limited.

According to some embodiments, the infusion pump may be capable of delivering fluid to one or more infusion sites. Such an arrangement may be desirable to reduce the time of infusion or administration and/or reduce the localization of the administered fluid. Accordingly, the infusion pump may be compatible with a needleset which may include any suitable number of needles to facilitate administration of a fluid. In some embodiments, the needleset may be bifurcated (for delivery to two infusion sites), trifurcated (for delivery to three infusion sites), quadfurcated (for delivery to four infusion sites), quintfurcated (for delivery to five infusion sites), or may have any other desirable number of needle hubs. It should be appreciated that the infusion pumps described herein may be compatible with any pre-existing needlesets, such as the KORU HIGH-Flo needleset.

In some embodiments, the infusion pump may be able to deliver fluid with a viscosity of 16 cP at up to 60 mL/hr/site through a 24 gauge or 27 gauge needleset. In some embodiments, the infusion pump may be able to deliver fluid through five 27 gauge needles of a needleset.

The inventors have also recognized the benefits of an infusion pump including a bubble trap that can be used to limit the delivery of air bubbles to an infusion site. The air bubbles may be formed as a result of frothing during syringe filling, or may be introduced into the fluid at any other point prior to infusion.

According to exemplary embodiments described herein, an infusion pump may include a bubble trap coupled to and/or in fluid communication with an outlet of a syringe at one end and a port at an opposing end, such that fluid flowing into and out of the syringe may pass through the bubble trap. The port may be connected to a vial when the infusion pump is used to extract fluid from the vial and fill the syringe, or may be connected to a needleset when the infusion pump is used to deliver fluid to an infusion site on a user. The bubble trap may be configured to permit fluid to pass through while limiting the number of air bubbles (or, in some embodiments, inhibit the passage of air bubbles) delivered to the infusion site with a combination of a gas-permeable hydrophobic membrane and a one-way check valve, as will be described in greater detail below. It should be appreciated that the bubble trap may be compatible with any of the fluid volumes/viscosities listed above.

In some embodiments, an infusion pump may be entirely mechanically operated. In some embodiments, an infusion pump may not include any electrically operated components. Accordingly, operation of the infusion pump may not be limited by the proximity of a power source (e.g., an electrical outlet). A user may operate the infusion pump at any suitable location. In some embodiments, the user may operate the infusion pump while in a mobile state. In addition, without the need for a battery to power the infusion pump, the user need not monitor a battery charge state or be concerned with recharging a battery.

In some embodiments, the infusion pump may include one or more wearable components (discussed in further detail below) to allow for hands-free operation of the infusion pump. It should be appreciated that instances in which different benefits are offered by the systems and methods disclosed herein are also possible.

In other embodiments, the primary functions of the infusion pump (syringe loading and/or drug delivery) may be mechanically operated, while other secondary functions of the infusion pump may be electrically operated and/or motorized (e.g., may require electrical power). In some embodiments, the limitation of electrical components may reduce the cost of manufacturing of an infusion pump, thereby reducing the cost of an infusion pump for the user, which may result in greater accessibility of the infusion pump.

In some embodiments, the infusion pump may be portable. For example, the infusion pump may either include a portable power source (e.g., a battery) or may not require any power sources to function, such that a user may operate the infusion pump without concern of a nearby charging outlet and/or port. In some embodiments, the infusion pump may be portable during an infusion process. The infusion pump may include one or more wearable accessories (e.g., straps, clips, etc.) to allow the user to move around without being hindered by the infusion pump. In some embodiments, the infusion pump may be used on a flat surface, such as a table-top or any other suitable surface.

In some embodiments, the infusion pump may include one or more components which may be in contact with the fluid. Accordingly, these one or more components may be disposable. For example, the infusion pump may include a disposable syringe, a disposable bubble trap, disposable tubing/connectors between various components, disposable needlesets, and/or any other disposable components. In some embodiments, all fluid-contacting components of the infusion pump may be silicone-free. In other embodiments, the infusion pump may include one or more components formed at least partially of silicone. In some embodiments, components formed at least partially of silicone may be in contact with fluid for less than 2 hours. In some embodiments, the fluid-contacting components of the infusion pump may be compatible with sensitive plasma-derived proteins. Of course, any suitable (e.g., compatible) material may be used within the infusion pump, as the present disclosure is not so limited.

In some embodiments, the infusion pump may be used to prime a needleset prior to infusion to allow a user to visually confirm fluid flow along the needleset. In some embodiments, the infusion pump may be used to check for blood backflow prior to infusion. Any of these verification processes (or any other suitable processes) may be conducted by operating one or more actuators of the infusion pump, which may simplify the process(es) for self-administering users.

In some embodiments, the infusion pump may include one or more features that provide a mechanical assist to decrease the required amount of input force from a user to operate. As will be described in greater detail below, the infusion pump may include one or more mechanical components which may amplify and/or modulate forces applied by the user to conduct one or more functions. For example, the infusion pump may convert a rotational torque applied to a lever to axial translation of a syringe plunger. In some embodiments, the infusion pump may require rotational torques less than 2 N m and forces less than 6 N to operate. Of course, the infusion pump may require any other suitable torque and/or force, as the present disclosure is not limited by the magnitude of input forces applied by the user.

In some embodiments, the infusion pump may convert a user input (e.g., rotation of a lever, rotation of a knob, sliding of a slider, etc.) into a substantially constant force applied to a syringe plunger. The substantially constant force applied to the syringe plunger may be related to the fluid viscosity, syringe size, needleset gauge, needleset length, needle diameter, number of infusion sites, intermediate tubing diameters, and/or any other relevant parameter. In some embodiments, the substantially constant force applied to the syringe plunger may be calibrated to a substantially constant fluid delivery flow rate, ranges of which have been described above. For example, an infusion pump using an 18″ needleset, a 0.7 mm inner diameter, and a 27 gauge diameter may convert a user input torque and/or force into about 15-75 N applied to the syringe plunger for delivering fluid flow rates between 5-60 mL/hr. In some embodiments, the substantially constant force applied to the syringe plunger may be at least 10 N, 12 N, 15 N, 20 N, 25 N, 30 N, 35 N, 40 N, 45 N, 50 N, 55 N, 60 N, 65 N, 70 N, 75 N, 80 N, 100 N, 125 N, 150 N, 175 N, 200 N, 300 N, and/or any other suitable force. In some embodiments, the substantially constant force applied to the syringe plunger may be less than or equal to 300 N, 200 N, 175 N, 150 N, 125 N, 100 N, 80 N, 75 N, 70 N, 65 N, 60 N, 55 N, 50 N, 45 N, 40 N, 35 N, 30 N, 25 N, 20 N, 15 N, 12 N, 10 N, and/or any other suitable force. Combinations of ranges for substantially constant force are also contemplated, including between 15 N to 60 N, 15 N to 75 N, 100 N to 200 N, 10 N to 200 N, and/or any other suitable combinations of substantially constant forces. Of course, other ranges, including ranges both greater than and less than those noted above are also contemplated as the present disclosure is not so limited. It should be appreciated that the infusion pump may be able to convert user input force to any suitable substantially constant force in order to deliver fluid to an infusion site.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

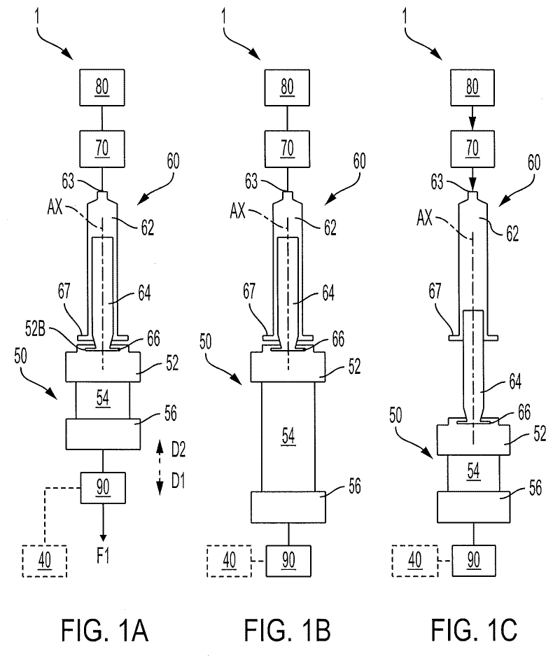

FIGS. 1A-1C show, according to some embodiments, a process of operating an infusion pump 1 to fill a syringe 60 with fluid contained in a vial 80. The infusion pump 1 includes a loading actuator 90 coupled to a loading assembly 50. The loading actuator 90 may also be coupled to a lock 40, such that operation of the lock 40 may temporarily fix (e.g., lock) the loading actuator 90 in position. As described previously, a bubble trap 70 may be positioned in between an outlet port 63 of the syringe 60 and the vial 80. In some embodiments, the bubble trap 70 may limit the transport of air bubbles across the bubble trap 70. As described in above, the vial 80 may contain any suitable fluid (e.g., medication).

In some embodiments, the syringe 60 may include a barrel 62 to hold a fluid (e.g., medication) and a plunger 64 to move the fluid through the barrel 62. The plunger 64 may be coaxially located within the barrel 62 and may be axially moveable relative to the barrel 62, such that movement of the plunger 64 in the proximal direction D1 along an axial direction AX may cause fluid to enter through an outlet port 63 and fill the barrel 62 and movement in the opposing distal direction D2 along the axial direction AX may expel fluid from the barrel 62 through the outlet port 63. The syringe 60 may also include a plunger flange 66. The syringe 60 described herein may be any standard syringe known in the art. For example, the syringe 60 may be a standard 60 mL syringe (Becton Dickinson, 309653).

As shown in FIGS. 1A-1C, the plunger flange 66 of the syringe 60 may be coupled to a holder 52 of the loading assembly 50 such that movement of the holder 52 may move the plunger flange 66 along the axial direction AX. The holder 52 may therefore be able to push or pull the plunger flange 66 along the axial direction AX. The plunger flange 66 may be removably coupled to the holder 52 to allow a user to replace the syringe 60. In some embodiments, the plunger flange 66 may fit within a slot 52B formed in the holder 52, as shown in FIGS. 1A-1C. The plunger flange 66 may be coupled to the holder 52 in any suitable manner to allow the two components to translate together.

The loading assembly 50 may further include a slider 56 coupled to the holder 52 via an energy storing member 54. In some embodiments, the energy storing member 54 may be a spring, as will be discussed in further detail below. Movement (e.g., linear translation) of the slider 56 may result in movement (e.g., linear translation) of the holder 52, which may in turn result in the movement of the plunger 64 in the axial direction AX. In some embodiments, the energy storing member 54 may modulate the rate of translation between the slider 56 and holder 52. For example, if the slider 56 is translated a distance rapidly between two positions, the energy storing member 54 may allow the holder 52 to travel a similar distance in a less rapid manner. In embodiments where the energy storing member 54 may be a spring, the rapid movement of the slider 56 stores energy within the energy storing member 54 (e.g., via elongation of a spring), which may then be slowly released (e.g., via contraction of the spring toward its original length) when translating the holder 52.

In some embodiments, the loading assembly 50 and syringe 60 may be axially fixed to one another. For example, the holder 52 and/or the slider 56 may be positioned on a rail (not shown), such that its movement may be confined to axial translation. In other embodiments, the loading assembly 50 (or one or more components of the loading assembly 50, e.g., the slider 56) may be positioned off-axially from the syringe 60. It should be appreciated that any component of the loading assembly 50 may be arranged in any suitable manner with respect to the syringe 60 such that movement of the slider 56 may result in translation of the holder 52, and subsequently translation of the plunger 64.

The slider 56 may be operated with a loading actuator 90, as shown in FIG. 1A, such that a user may operate the loading actuator 90 to move the slider 56. As described above, movement of the slider 56 may result in loading/expelling fluid from the syringe 60. The loading actuator 90 may be a lever, a knob, a button, a rotary dial, a slider, or any other suitable user-operable structure. In some embodiments, the loading actuator 90 may only be operable in one direction. For example, the loading actuator 90 may include a directional lock 40, which may engage with the loading actuator 90 to allow movement of the loading actuator 90 in one direction and prevent movement in the opposing direction. In some embodiments, the directional lock 40 may include a ratchet arrangement. The directional lock 40 may be selectively engaged with the loading actuator 90, such that disengagement between the actuator 90 and the lock 40 may allow the actuator 90 to move in any suitable direction. As will be described in further detail below, the directional lock 40 may be operable via an actuator, which may allow a user to engage or disengage the lock 40 from the loading actuator 90.

As described previously, a syringe plunger 64 may be axially moveable with respect to a syringe barrel 62. In some embodiments, the syringe barrel 62 may be fixed in place. In some embodiments, the loading actuator 90 may be biased towards the syringe 60 in the distal direction D2, such that absent an external force (e.g., a user operating the actuator 90), the loading actuator 90 may urge the slider 56, and subsequently the holder 52 (via the energy storing member 54) and the plunger 64 towards the outlet port 63, as shown in FIG. 1A. In some embodiments, the directional lock 40 may counteract the bias of the loading actuator 90 so that the loading actuator 90 may be moveable against the direction of its bias. As will be described in great detail below, in some embodiments, the loading actuator 90 may be biased with a second energy storing member.

In some embodiments, in operation, a user may fill a syringe 60 with fluid from a vial 80 by first attaching a bubble trap 70 to an outlet port 63 of the empty syringe 60 and subsequently loading the bubble trap and syringe assembly within an infusion pump 1, ensuring that the plunger flange 66 is coupled to the holder 52. In some embodiments, the bubble trap 70 may be attached to the syringe 60 after the syringe 60 has been loaded into the infusion pump 1. The user may then load the vial 80 (which may include any suitable fluid, e.g., medication) into the infusion pump 1 through any suitable connector. In some embodiments, the vial 80 may be loaded into the infusion pump 1 via a spike connector, such that attachment of the vial 80 to the connector may pierce the vial 80 to allow fluid flow out of the vial 80. Accordingly, in some embodiments, the spike connector may be removably coupled to the vial 80 and/or an outlet port of the infusion pump. In some embodiments, the spike connector may be vented. The infusion pump 1 may include any number of couplings (e.g., a Luer-lock hub positioned by the outlet port) to ensure suitably fluid-tight connections between the syringe 60, bubble trap 70, and vial 80.

The user may then engage a directional lock 40 with a loading actuator 90 to prohibit movement of the loading actuator 90 in at least one direction (e.g., in the direction of the loading actuator bias). The user may subsequently apply a force F1 to the loading actuator 90 in a direction opposite of the loading actuator bias, as shown in FIG. 1A. In some embodiments, force F1 may correspond to a desired volume of fluid to be loaded into the syringe 60. A slider 56 may be coupled to the loading actuator 90 such that actuation of the loading actuator 90 may result in movement (e.g., axial translation) of the slider 56, as shown in FIG. 1B. It should be appreciated that engagement of the directional lock 40 and the loading actuator 90 may fix the loading actuator 90 (and in some embodiments, the slider 56) in place after actuation.

In some embodiments, axial translation of the holder 52 may be limited by surface tension of fluid exiting the vial 80, passing through the bubble trap 70, and entering the barrel 62, as well as stiction between the plunger 64 and the barrel 62 (e.g., the plunger may include a rubber tip positioned at an opposing end to the flange 66, which may interact frictionally with the inner walls of the barrel 62). In some embodiments, the stiffness of the energy storing member 54 may be less than the fluid resistance force from the surface tension of the fluid flowing through the various components and syringe barrel/plunger stiction, such that movement of the slider 56 away from the holder 52 initially causes the energy storing member 54 to elongate first, prior to movement of the holder 52 toward the slider 56. Locking the loading actuator 90 in a proximal position (e.g., as shown in FIG. 1B) may tension the energy storing member 54 between the slider 56 (which may be in a proximal position) and the holder 52, which may still be in its original distal position. The energy storing member 54 may then slowly release its stored energy and move the holder 52 to a proximal position, as shown in FIG. 1C. Axial translation of the holder 52 in the proximal direction D1 may result in movement of the plunger 64 to a proximal position, which may urge fluid to flow from the vial 80 to the syringe 60, as depicted by the arrows between vial 80, bubble trap 70, and outlet port 63 in FIG. 1C. It should be appreciated that the movement of the holder 52 may be controlled by both the stiffness of the energy storing member 54 and the aforementioned fluid transfer properties noted above.

In some embodiments, fluid flow between the vial 80 and the barrel 62 may stop once the holder 52 reaches its proximal most position (e.g., wherein the energy storing member 54 is no longer under tension). The user may then remove the vial 80 from the infusion pump 1, and replace with a new vial, if desired. Operation of the loading actuator 90 may be repeated as many times as desired to fill the syringe 60 with a suitable volume of fluid. In each loading process, the loading actuator 90 may be actuated by a magnitude which corresponds to the volume of the vial 80. The infusion pump 1 may include a visual indicator to allow the user to calibrate the actuation force (e.g., force F1) with the desired filling volume of the syringe 60, as described in further detail below. In embodiments where the syringe 60 volume capacity is equivalent to a single vial 80, the loading process may only need to take place once.

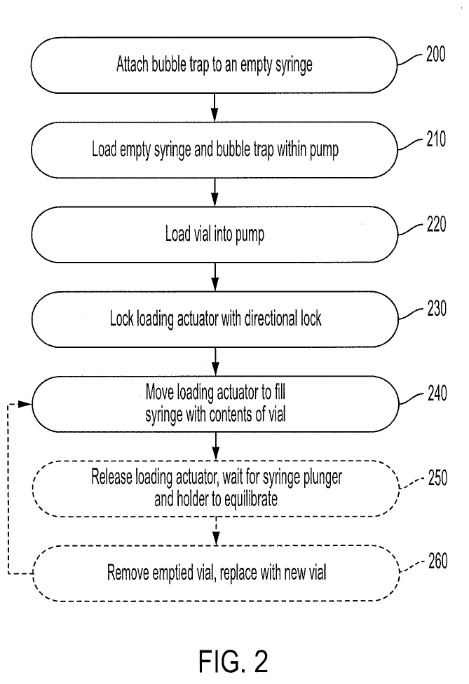

FIG. 2 shows, according to some embodiments, a flow chart for a method of operating an infusion pump to fill a syringe. In block 200, a bubble trap is attached to and fluidically connected to an outlet port of an empty syringe. In block 210, the syringe and attached bubble trap are loaded within the infusion pump. In some embodiments, the order of operation for blocks 200 and 210 may be reversed. In block 220, a vial is loaded into the infusion pump. In some embodiments, loading the vial (which may contain medication) into the pump may fluidically connect the outlet port of the syringe, the bubble trap, and the vial. As shown in block 230, a loading actuator may then be locked with a directional lock to prevent movement of the loading actuator in at least one direction. The loading actuator may engage with the directional lock in any suitable manner to directionally limit movement of the loading actuator. In block 240, the loading actuator may be actuated in order to fill the syringe with fluid from the vial. As described in greater detail above, movement of the loading actuator may result in axial movement of a plunger of the syringe in a proximal direction away from a barrel of the syringe, urging fluid to flow from the vial into the syringe barrel. Movement of the loading actuator may also cause loading of the energy storing member 54, e.g., a spring that is elongated.

In block 250, the user may let go of the loading actuator and wait for a desired volume of fluid to flow from the vial into the syringe. It should be appreciated that in embodiments where the directional lock is engaged with the loading actuator, the loading actuator may remain stationary after being released by the user, despite being biased in an opposing direction. Accordingly, the user may not be required to operate the infusion pump in block 250. In block 260, the user may remove the emptied vial from the infusion pump and optionally replace with a new vial. The process of blocks 240-260 may be repeated any number of times to fill the syringe with a desired volume of fluid.

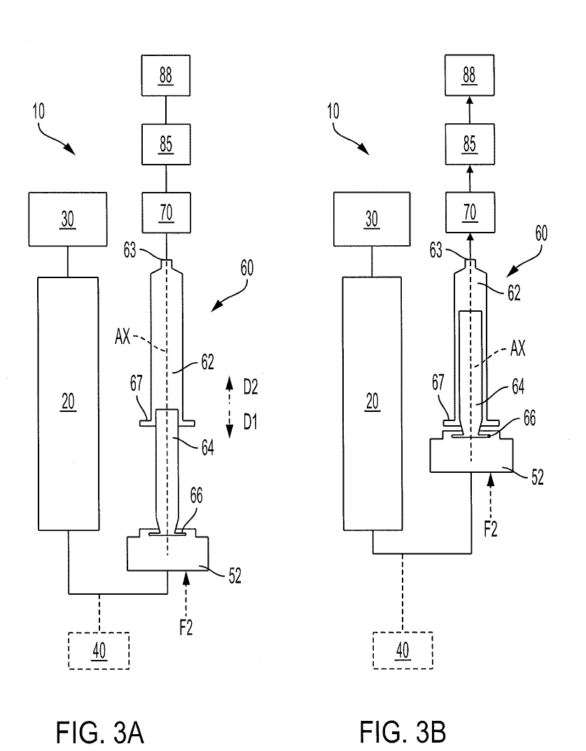

FIGS. 3A-3B show, according to some embodiments, a process of operating an infusion pump 10 to expel fluid from a syringe 60 to a needleset 85. In some embodiments, the needleset 85 may be connected to an infusion site 88, such that operating the infusion pump 10 may facilitate infusion of the fluid into the infusion site 88. The needleset 85 may be fluidically connected to an outlet port of the syringe through a bubble trap 70, which may reduce the likelihood of bubbles delivered to the patient. The syringe 60 of FIGS. 3A-3B may be similar to one described in accordance with infusion pump 1 from FIGS. 1A-1C. Accordingly, syringe 60 may include a plunger 64 coaxially located within a barrel 62, such that axial displacement of the plunger 64 within the barrel may fill or expel fluid from the barrel 62. As described previously, the syringe 60 may include a plunger flange 66 coupled to a holder 52, such that movement (e.g., axial translation, pushing, or pulling along the axial direction AX) of the holder 52 may directly correspond to movement of the plunger flange 66, and subsequently the plunger 64. The holder 52 may be considered a contact surface which may contact the plunger 64 (e.g., through plunger flange 66).

In some embodiments, the holder 52 of infusion pump 10 may be coupled to an energy storing assembly 20 configured to apply a substantially constant force F2 to the holder 52, as shown in FIGS. 3A-3B, and subsequently the plunger 64. This substantially constant force F2 may result in a substantially constant flow rate of fluid out of the outlet port 63 of the syringe 60. The infusion pump 10 may also include a rate actuator 30 to allow a user to control the magnitude of the substantially constant force F2. Mechanical coupling between the rate actuator 30 and the energy storing assembly 20 may enable adjustment of the substantially constant force F2 via non-electric control of the rate actuator 30. In some embodiments, the rate actuator may mechanically adjust the magnitude of the substantially constant force. For example, the rate actuator 30 may be a lever, a knob, a button, a rotary dial, a slider, or any other suitable user-operable structure. Of course, electrical embodiments of a rate actuator are also contemplated.

In some embodiments, the infusion pump 10 may also include a directional lock 40. The directional lock 40 may be similar in operation to that described in accordance with infusion pump 1. Namely, the directional lock 40 may be engaged to lock or temporarily fix the holder 52 to prevent axial translation in at least one direction. In some embodiments, the directional lock 40 may be operated when flow fluid out of or into the infusion pump 10 are undesirable. For example, the directional lock 40 may be engaged to stop fluid flow out of the syringe 60 in the middle of an infusion process. In another example, the directional lock 40 may be engaged to stop fluid flow out of the syringe 60 prior to operation. The directional lock 40 may include an actuator operable by a user to operate the lock 40. In some embodiments, the actuator may operate as an on/off toggle controlling fluid flow out of the infusion pump 10, as will be described in further detail below.

It should be appreciated that processes involving delivery of fluid out of the syringe 60 may begin with the syringe 60 containing a desired volume of fluid. A method of filling a syringe with fluid according to some embodiments is described in greater detail above. Accordingly, as shown in FIG. 3A, delivery of fluid out of the syringe 60 may begin when the plunger 64 is spaced from the outlet port 63 to accommodate for fluid (e.g., medication) contained within the barrel 62.

In operation, a user may deliver fluid from a syringe 60 to a patient via a needleset 85 by first positioning the syringe plunger 64 in a distal position, as shown in FIG. 3A. In some embodiments, this process involves loading the syringe 60 with fluid by either the methods described above (e.g., via a loading assembly 50 and loading actuator 90, as shown in FIGS. 1A-1C) or manually. A directional lock 40 may be engaged to prevent fluid flow out of the barrel 62 prior to operation. The user may then install one end of a needleset 85 on the infusion pump 10. In some embodiments, the user may choose to prime the needleset 85 prior to insertion into a desired infusion site. For example, the user may operate a rate actuator 30 to its lowest possible setting, unlock the directional lock 40 until fluid is observed to be moving along the needleset 85 towards a needle tip. The user may then place a second end of the needleset 85 on the desired infusion site 88 (e.g., a patient). In some embodiments, the user may reverse fluid flow using an additional actuator (e.g., a loading actuator described previously) in order to check for blood backflow from the desired infusion site. This process may ensure that the needleset 85 is positioned in a suitable infusion site 88 on the patient and/or user. The user may then operate a rate actuator 30 to adjust the flow rate of fluid out of the syringe 60 according to a desired fluid infusion rate. The rate actuator 30 may include visual indicators to allow the user to adjust the flow rate to a desired value. The user may then release the directional lock 40 to allow fluid to flow at the adjusted flow rate from the syringe 60 into the infusion site. As indicated by the arrows in FIG. 3B, disengagement of the directional lock 40 may allow the energy storing assembly 20 to apply a constant force F2 (corresponding to the desired flow rate selected by the user with the rate actuator 30) to the holder 52 and subsequently the plunger 64, resulting in fluid flow from the outlet port 63 to the needleset 85.

In some embodiments, the user may choose to adjust the flow rate at any point during the infusion process. It should be appreciated that the adjustment in flow rate may be continuous (e.g., not discretized), such that minute adjustments may be made for comfort or other suitable reasons. As described earlier, the user may also stop fluid delivery at any point during the infusion process by engaging the directional lock 40.

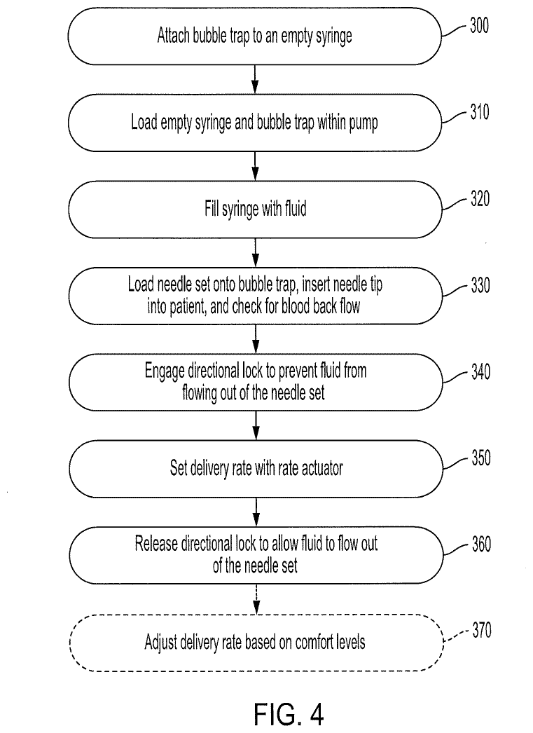

FIG. 4 shows, according to some embodiments, a flow chart for a method of operating an infusion pump to deliver fluid. In block 300, a bubble trap is attached to and fluidically connected to an outlet port of an empty syringe. In block 310, the syringe and attached bubble trap are loaded within the infusion pump. In some embodiments, the order of operation for blocks 300 and 310 may be reversed. In block 320, the syringe may be filled with fluid (e.g., medication) using any suitable method. In some embodiments, block 320 may represent the syringe filling methods depicted in FIG. 2. As shown in block 330, a needleset may be fluidically connected to the bubble trap and subsequently inserted into a desired infusion site on the patient. The position of the needleset on the patient/user may be verified by checking for blood backflow, as will be described in further detail below. In some embodiments, the needleset may be primed prior to insertion into the desired infusion site, through a process that will be described in further detail below. In block 340, a directional lock may be engaged to prevent fluid from flowing out of the syringe into the needleset prior to desired operation of the infusion pump. In block 350, the delivery rate (e.g., fluid flow rate from the syringe) may be adjusted by manipulation of a rate actuator. In block 360, the directional lock may be released to allow fluid to flow out of the needleset and into the patient. It should be appreciated that the directional lock may be engaged at any point during the infusion process to stop fluid flow into the patient. In some embodiments, as depicted in block 370, the user may adjust the delivery rate (e.g., fluid flow rate) to increase comfort or for any other suitable purpose. The delivery rate may be adjusted by a rate actuator, as described previously.

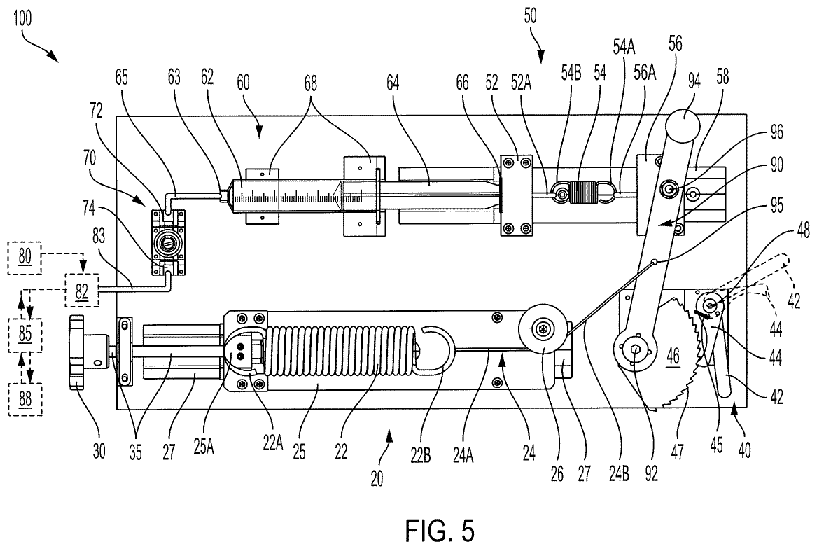

FIG. 5 shows, according to some embodiments, an infusion pump 100 for filling a syringe 60 with fluid and delivering said fluid to an infusion site 88 at an adjustable but constant flow rate. In some embodiments, the infusion pump 100 may operate purely mechanically, without any electrical components. In other embodiments, the syringe filling and fluid delivery features of the pump may be mechanical while other features of the pump may be electrical. It should be appreciated that the non-electrical nature of the syringe filling and fluid delivery features of the infusion pump may result in a low-cost and robust infusion pump. In some embodiments, the infusion pump described herein may be used in environments without readily accessible electricity.

As shown in FIG. 5, an infusion pump 100 may include several of the features previously described. For example, the infusion pump 100 may include a rate actuator 30 configured to control the magnitude of a substantially constant force applied from an energy storing assembly 20 to a loading assembly 50. A portion of the loading assembly 50 (e.g., a holder 52) may be configured to transfer the substantially constant force to a plunger 64 of a syringe 60, which may subsequently urge fluid contained within the syringe to flow out of the syringe 60. In some embodiments, the syringe 60 may be filled with a fluid (e.g., medication) contained within a vial 80 with the assistance of the infusion pump 100 when a user operates a loading actuator 90. In some embodiments, the syringe 60 may be connected to a vial 80 or a needleset 85 through a bubble trap 70. The bubble trap may be configured to prevent delivery of bubbles to the infusion site 88, as described in further detail below. The infusion pump 100 may also include a directional lock 40 and an associated locking actuator 42 for operating the lock 40.

As shown in FIG. 5, in some embodiments, the energy storing assembly 20 may include a delivery spring 22, a connector wire 24 (e.g., cable), and a roller 26. The energy storing assembly 20 may be loaded on a sledge 25 which may be slidable on a track 27. A first end 22A of the spring 22 may be coupled to a first end 25A of the sledge, while a second end 22B of the spring 22 may be coupled to a first portion 24A of the connector wire 24. In some embodiments, the first end 22A and second end 22B of the spring 22 may be hook shaped (as shown in FIG. 5), such that the ends may hook onto features such as the first end 25A of the sledge or the connector wire 24. The connector wire 24 may be fixed to a different component of the infusion pump 100, as will be described in further detail below. In some embodiments, the connector wire 24 may be secured to the second end 22B of the spring 22 to allow efficient force transfer between the two components. Of course, other embodiments of the spring 22 and related attachment schemes to the sledge 25 and/or connector wire 24 may also be employed, as the present disclosure is not so limited.

As described previously, the sledge 25 may be slidable on the track 27. In some embodiments, the sledge 25 may be connected to the rate actuator 30 via a lead screw 35. The rate actuator 30 may be rotatably coupled to the lead screw 35, such that rotation of the rate actuator 30 may result in rotation of the lead screw 35, which in turn, may cause axial translation of the sledge 25 along the track 27. Accordingly, the rate actuator 30 may be used to translate the sledge 25 along the track 27 in any desired direction. Although a rotatable rate actuator 30 is shown in FIG. 5, any other suitable rate actuator 30 may be used to operate the sledge 25. It should be appreciated that any suitable arrangement of the sledge 25 and rate actuator 30 which converts operation of the rate actuator 30 into translation of the sledge 25 may be employed.

In some embodiments, the stored energy within the energy storing assembly 20 may be adjusted by operation of the rate actuator 30. For example, in embodiments where the energy storing assembly 20 includes a spring 22, operation of the rate actuator 30 (e.g., rotation of the knob shown in FIG. 5) in one direction may result in extension of the spring 22 and operation of the rate actuator 30 in an opposite direction may result in contraction of the spring 22. As the connector wire 24 may be fixed to the loading actuator 90 at one end and coupled to the spring 22 at another end (e.g., at second end 22B of the spring 22), movement of the sledge 25 (and its first end 25A) away from the loading actuator end of the connector wire 24 may result in the extension of the spring 22. It should be appreciated that the tensile stiffness of the connector wire 24 may be sufficiently greater than the stiffness of the spring 22, such that the length of the connector wire 24 may remain constant during operation of the infusion pump 100. In some embodiments, the connector wire may be substantially inflexible relative to the spring 22. In other words, the connector wire 24 may not deform with movement of the sledge 25.

It should be appreciated that the connection mechanism between the sledge 25 and the rate actuator 30 may be sufficiently robust to avoid movement (e.g., translation) of the sledge on the track 27 without direct operation of the rate actuator 30. In other words, the maximum tension in the spring 22 may not be sufficient to overcome the connection between the rate actuator 30 and the sledge 25 to move the sledge 25 without user interference.

In some embodiments, the closer the second end 22B of the spring 22 is positioned to the roller 26, the greater the extension of the spring 22. For example, when the second end 22B of the spring 22 is positioned closer to the roller 26, the spring 22 may be under greater tension than when the second end 22B is positioned further away from the roller 26. Accordingly, the closer the second end 22B is positioned to the roller 26, the greater its stored energy. For example, FIG. 5 shows a spring 22 in a generally contracted configuration with the second end 22B positioned away from the roller 26.

As described previously, a portion of the connector wire 24 may be coupled to the second end 22B of the spring 22, as shown in FIG. 5. The connector wire 24 may further be partially wrapped around the roller 26, and connected to an anchor point 95 of the loading actuator 90. Accordingly, the roller 26 may act as a pulley for the connector wire 24. The loading actuator 90 may include a pivot point 92 around which it may rotate. The pivot point 92 may be offset from the anchor point 95 along the loading actuator 90, such that rotation of the loading actuator 90 around its pivot point 92 may change the length of the second portion 24B of the connector wire 24. As described previously, the connector wire 24 may be sufficiently rigid, such that the total length of the wire 24 (including the first portion 24A and second portion 24B) may be substantially constant during operation. Accordingly, the connection between the connector wire 24 and the rigid loading actuator 90 may act as a linkage between the spring 22 (or other suitable energy storing member) and the loading assembly 50. It should be appreciated that the connector wire 24 may be under tension regardless of the position of the sledge 25, such that the loading actuator 90 may be biased towards the spring 22.

In some embodiments, the loading actuator 90 may be fixed to a slider 56 of the loading assembly 50, as shown in FIG. 5. The slider 56 and the loading actuator 90 may be fixed at an attachment point 96, such that connection between the slider 56 and loading actuator 90 may not impede the rotation of the loading actuator 90 around its pivot point 92. Accordingly, the slider 56 may be slidable along a track 58, such that rotation of the loading actuator 90 may result in axial translation of the slider 56. In some embodiments, the loading actuator 90 may also include a user-operated handle 94 to allow a user to operate the loading actuator 90.

As described previously, in some embodiments, the slider 56 may be connected to a holder 52 via an energy storing member 54 (e.g., a spring, as shown in FIG. 5). The slider 56 may include a hook 56A or any other suitable structure to engage a portion 54A of the energy storing member 54. Similarly, the holder 52 may include a hook 52A or any other suitable structure to engage a portion 54B of the energy storing member 54. It should be appreciated that any suitable connection means between the slider 56, energy storing member 54, and holder 52 may be used, as the present disclosure is not so limited. In some embodiments, the holder 52 may also be slidable along the track 58 such that translation of the slider 56 on the track 58 may cause translation of the holder 52 via the energy storing member 54. As described previously, the holder 52 may be configured to hold a plunger flange 66 of the syringe 60. In some embodiments, the plunger flange 66 may be removably inserted into a slot formed in the holder 52. It should be appreciated that any suitable loading of the plunger flange 66 into the holder 52 to sufficiently couple the axial translation of the two components may be employed. In other words, the holder 52 may be able to push and/or pull the plunger 64.

In some embodiments, the barrel 62 of the syringe 60 may be fixed in place with one or more holders 68, as shown in FIG. 5. It should be appreciated that any suitable structure may be used to fix the barrel 62 relative to the plunger 64 of the syringe 60. For example, in some embodiments, the infusion pump may include a housing arrangement configured to confine the position of the barrel 62.

The syringe 60 may include an outlet port 63 in fluid communication with a bubble trap 70 and subsequently, a connector 82. In some embodiments, the connector 82 may be configured to couple to a vial 80 for filling the syringe 60 and/or a needleset 85 for delivering fluid out of the syringe 60. The outlet port 63 may be connected to a first port 72 of the bubble trap 70 via a connection 65. In some embodiments, the connection 65 may be tubing, although any other suitable fluid-tight connection between the outlet port 63 and the bubble trap 70 may be employed. Similarly, the connector 82 may be connected to the bubble trap via a connection 83, which may, in some embodiments, be fluid-tight tubing. It should be appreciated that the present disclosure is not limited by the connection means between the syringe 60, bubble trap 70, and/or connector 82.

In some embodiments, the infusion pump 100 may also include a directional lock 40 which may limit movement (e.g., rotation) of the loading actuator 90 in at least one direction. The directional lock 40 may include a ratchet wheel 46, an actuator 42, and a pawl 44. The actuator 42 may be user operable, such that a user may choose to lock or unlock the lock 40 using the actuator 42. The locking actuator 42 may be a lever, a knob, a button, a rotary dial, a slider, or any other suitable user-operable structure. In some embodiments, the actuator 42 may act as a toggle to operate the lock 40. The ratchet wheel 46 may be rotationally coupled to the loading actuator at the pivot point 92. Accordingly, when the ratchet wheel 46 is blocked from rotating (as will be described below), the loading actuator 90 may also be blocked from movement.

As shown in FIG. 5, when the actuator 42 is in a first position (solid lines), the pawl 44 may be engaged with the ratchet teeth 47 of the ratchet wheel 46. In some embodiments, the ratchet teeth 47 may be angled in a manner that allows the pawl to move along the ratchet teeth 47 in one direction, but not the other. For example, each ratchet tooth 47 may include a substantially angled face and a substantially vertical face, enabling movement of the pawl in the direction of the angled faces and limiting movement in the opposing direction. This limitation may be facilitated by employing a spring 45 between the actuator 42 and the pawl 44. The spring 45 may limit the rotation of the pawl 44 around its pivot point 48 such that the pawl 44 may not be able to traverse the substantially vertical faces when the actuator 42 is in the first position (solid lines). Accordingly, the directional lock 40 may be activated when the pawl 44 is engaged with the ratchet wheel 46, effectively blocking movement of the loading actuator 90. The actuator 42 may be moveable to a second position (dashed lines) in which the pawl 44 is released from the ratchet wheel 46. Accordingly, the ratchet wheel 46 may be free to rotate along with the loading actuator 90 around pivot point 92 when the directional lock 40 is unlocked.

Of course, other locking arrangements for the directional lock 40 are also contemplated. For example, rotation of the loading actuator may be permitted/limited by a friction brake and pad (or any other frictional engagement, e.g., a friction pad such as a shoe), such that toggling the locking actuator between its locked and unlocked configurations may frictionally engage or disengage the friction brake from its corresponding pad (e.g. a shoe), effectively blocking or unblocking rotation of the loading actuator. Engagement between the friction brake and pad may generate enough friction to temporarily inhibit rotation of the loading actuator.

Although FIG. 5 shows the sledge 25 positioned parallel to the loading assembly 50 and syringe 60, it should be appreciated that any suitable arrangement of the various components of the infusion pump 100 may be employed, and may bear little resemblance to the arrangement depicted in FIG. 5. Accordingly, the present disclosure is not limited by the spatial arrangement of the components depicted in FIG. 5. In some embodiments, the various components of the infusion pump 100 may be arranged to achieve a compact footprint of the infusion pump 100.

It should be appreciated that the greater the extension of the spring 22 (e.g., when the second end 22B of the spring 22 is positioned close to the roller 26 by operation of the rate actuator), the greater the force which may be transferred from the spring 22 to the loading actuator 90. Accordingly, the rate actuator 30 may control the rate of fluid flow out of the syringe 60 by controlling the extension of the spring 22. As described earlier, adjusting the extension of the spring 22 may result in a change in the force exerted by the spring 22 on the loading actuator 90, which may subsequently result in a greater force exerted by the loading actuator 90 on the holder 52. The greater the tension (e.g., stored clastic energy) in the spring 22, the greater the force which may be transferred to the plunger 64, resulting in a greater fluid flow rate out of the syringe 60.

In operation, a user may begin an infusion process by first attaching a bubble trap 70 to an empty syringe 60, and inserting the assembly into the infusion pump 100. The user may then attach a connector 82 to the infusion pump 100, which may subsequently be coupled to a vial 80 filled with fluid (e.g., medication). In this way, the vial 80, connector 82, bubble trap 70, and syringe 60 (through outlet port 63) may be in fluid communication. As described previously, the connector 82 may include a spike to pierce a cap or seal of the vial 80 in order to open fluid communication into the vial. At this point, even with the vial 80 in fluid communication with the syringe 60, in some embodiments, fluid does not flow from the vial into the syringe until the syringe plunger 64 is pulled back by operating the loading actuator 90.

To help decrease the amount of force needed to operate the loading actuator 90 in order to fill the syringe, in some embodiments, the user may decrease the biasing force of the spring 22 acting on the loading actuator 90 by setting the rate actuator 30 to the lowest possible setting. Doing so may move the spring 22 to a position that is closer to the loading actuator 90, which may reduce the bias on the loading actuator 90 from the spring 22.

Prior to operating the loading actuator 90, a user may engage the directional lock 40 by locking the actuator 42. In this way, the ratchet 46 may rotate in one direction (e.g., clockwise around pivot point 92, as shown in FIG. 5), but may be limited in the opposing direction. The directional lock 40 may be sufficiently robust to counteract any bias applied to the loading actuator 90 by the spring 22 and rotationally lock the loading actuator 90 in place. The user may then operate the loading actuator 90 by the handle 94 corresponding to the desired filling volume of the syringe 60. In some circumstances, a single vial 80 may suffice for an infusion process, whereas in other situations, more than one vial 80 may be necessary. It should be appreciated that the syringe 60 may be selected with the desired volume of fluid (e.g., medication) in mind. In some embodiments, operation of the loading actuator 90 may involve rotation of the actuator 90 around its pivot point 92. Since the directional lock 40 may be engaged at this point, the user may release the loading actuator 90 after a desired degree of rotation. The directional lock 40 may retain the loading actuator 90 in place even after the user has released the handle 94. In some embodiments, the infusion pump may include visual markers to indicate the fill volume associated with various positions of the handle 94.

As described previously, the slider 56 may be coupled to the loading actuator 90 such that rotation of the handle 94 may result in direct linear translation of the slider 56 on track 58, as shown in FIG. 5. However, it should be appreciated that the holder 52 (directly coupled to the plunger 64) is not directly linearly coupled to the slider 56 in some embodiments. Accordingly, operation (e.g., clockwise rotation) of the loading actuator 90 may not immediately translate the holder 52 along the track 58. Instead, in some embodiments, the intermediate energy storing member 54 may modulate the force between the slider 56 and holder 52, such that the holder 52 may translate along the track 58 more slowly. In this way, the user may rapidly operate the loading actuator 90, causing the energy storing member 54 to elongate and store potential energy, release the loading actuator 90, and leave the infusion pump 100 to automatically fill the syringe 60 as the energy storing member 54 slowly contracts (e.g., releasing stored energy from its elongated state) between the holder 52 and slider 56. It should be appreciated that, due to the connection of the loading actuator 90 and the slider 56 at attachment point 96 (as shown in FIG. 5), the slider 56 may also be fixed in place when the directional lock 40 is engaged. Accordingly, the slow contraction of the energy storing member 54 may only be able to translate the holder 52 along track 58, resulting in fluid flow from the vial 80 to the barrel 62, effectively filling the syringe 60. It should be appreciated that the rate of contraction of the energy storing member 54 may be limited by its stiffness, stiction between the plunger 64 and barrel 62, fluid viscosity and surface tension, and/or any other suitable parameter. The term “slow” used herein is not an indicator of any particular contraction rate of the energy storing member 54 or flow rate into outlet port 63, but rather a relative term used to describe the movement of holder 52 as compared to the rate by which the slider 56 moves due to the user moving the handle 94. It should be appreciated that the holder 52 may not necessarily move slower than the slider 56 during a syringe filling process. In some embodiments, the holder may move at the same rate as, or faster than, the movement rate of the slider.

In some embodiments, when the energy storing member 54 reaches its contracted configuration, wherein the holder 52 is positioned near the slider 56, the filling process may be completed as depicted in FIGS. 1B and 1C. At this point in operation of the infusion pump 100, the user may continue filling the syringe 60 by following the processes described above, or may prepare for infusion. It should be appreciated that the syringe 60 may be filled with any suitable number of vials containing a total volume equal to the barrel 62 capacity. In some embodiments, the directional lock 40 may be engaged during the entirety of the filling process to prevent premature outflow of fluid from the barrel 62.

Once the syringe 60 is filled with a sufficient volume of fluid (e.g., medication), the user may prepare for the infusion process. The emptied vial 80 may be removed from the connector 82 and disposed of appropriately. The user may then attach a needleset 85 to the connector 82 in a fluid-tight manner (e.g., using a luer lock connection). As described previously, the needleset 85 may be coupled to the connector 82 at one end a priming process may take place. In some embodiments, a user may prime the needleset 85 by first setting the rate actuator 30 at its lowest possible setting and unlocking the directional lock 40 to allow fluid to flow out of the pump 100 and into the needleset 85, towards a needle tip. Once proper flow has been observed, the user may then insert a second end of the needleset 85 into an infusion site 88 on a patient. Once the needleset 85 is inserted into the appropriate infusion site, a user may verify the location of the infusion site by checking for blood backflow. In some embodiments, the user may move the loading actuator 90 slightly as if to fill the syringe 60 (e.g., rotate in a clockwise direction with respect to FIG. 5). The user may visually inspect the tubing of the needleset 85. In some embodiments, if the user observes blood within the tubing of the needleset 85, the user may remove the needleset 85 from the infusion site and replace the needleset with a new needleset. If the user does not observe any blood, then the insertion of the needleset 85 may have been successful, and the infusion process may begin.

To begin delivery of fluid from the syringe 60 to the infusion site 88, the user may adjust the rate actuator 30 to a desired fluid flow rate. The rate actuator 30 may include visual markers to indicate the range of selectable fluid flow rate. It should be appreciated that the rate actuator 30 may be continuously operable, such that the user may select any desired fluid flow rate within the acceptable range determined by the translation distance of the sledge 25. In other words, the user may be able to precisely select a fluid flow rate based on comfort, timing, or any other suitable reason. In some embodiments, the acceptable range of fluid flow may be limited by acceptable flow rates as determined by the FDA (or any other regulatory agencies), medical professionals, the manufacturers of the medication, and/or any other suitable reason.

As discussed previously, adjustment (e.g., rotation) of the rate actuator 30 (e.g., a knob) may result in linear translation of the sledge 25. Accordingly, the spring 22 may be extended based on the degree of adjustment, such that a significantly extended spring 22 may correspond to a greater fluid flow rate out of the syringe 60 compared to a partially extended spring 22. It should be appreciated that adjustment of the spring 22 tension (via the rate actuator 30) may be conducted while the directional lock 40 is locked. In this way, the loading actuator 90 (and associated loading assembly 50 and syringe plunger 64) may remain stationary during the adjustment process, preventing premature fluid outflow from the syringe 60.

Upon selection of a suitable flow rate with the rate actuator 30, the user may release the directional lock 40 to allow fluid to flow out of the syringe 60 at a flow rate commensurate with the selection on the rate actuator 30. In some embodiments, a user may elect to prime the needleset 85 prior to inserting the needleset 85 into the infusion site. The user may then check for blood backflow and subsequently select a suitable flow rate with the rate actuator 30. The user may visually inspect the needleset 85 to track the fluid from the syringe 60 as it flows towards the infusion site 88. The user may then lock the directional lock 40, and operate the loading actuator 90 as described previously to check if the needleset 85 was inserted into/near a blood vessel. Once the user confirms that the needleset 85 was installed properly (e.g., no blood outflow from infusion site 88 and into tubing 83), the directional lock 40 may be unlocked to restart infusion.

In operation, unlocking the directional lock 40 with locking actuator 42 may disengage pawl 44 from the ratchet wheel 46, as shown in dashed lines in FIG. 5. Accordingly, the loading actuator 90 may be free to rotate around its pivot point 92 in any direction. Given the tension in the connector wire 24 and spring 22, the loading actuator 90 may be biased or urged in the direction of fluid outflow (e.g., counterclockwise rotation in FIG. 5) upon release of the directional lock 40. The loading actuator 90 may then attempt to minimize its bias by rotating around its pivot point 92, which may decrease the length of the second portion 24B of the connector wire. In this way, as the spring 22 contracts, releasing its stored energy, the moment arm around the pivot point 92 of the loading actuator 90 increases. Accordingly, although the spring 22 may apply a linearly decreasing force to the loading actuator 90 as it contracts, the linkage of the loading actuator 90, roller 26, and connector wire 24 may convert the linearly variable force to a substantially constant force applied to the holder 52. In some embodiments, the linkage may amplify the linearly decreasing force from the spring 22 such that a substantially constant force is applied to the holder 52. It should be appreciated that any other suitable arrangement of the components described herein, configured to convert a variable force to a constant force, may be employed, as the present disclosure is not so limited. For example, in some embodiments, an infusion pump may be configured to output a single, non-adjustable, but constant flow rate from the syringe. In these embodiments, the spring location may be fixed (e.g., there may not be a rate actuator to mechanically displace the spring), such that the spring may only exert a single constant force to the syringe.

During fluid delivery, tension within the spring 22 and connector wire 24 work to move (e.g., rotate) the loading actuator 90 towards the plunger 64 in order to pressurize fluid within the barrel 62 to cause fluid outflow out through the outlet port 63 and into the infusion site 88. It should be appreciated that the energy storing member 54 may limit the speed of movement of the holder 52 with respect to the slider 56 in its tensioned state. In other words, when the energy storing member 54 is compressed, it may act as a solid, non-deforming body. Accordingly, the energy storing member 54 may simply transfer the axial displacement of the slider 56 to the holder 52 with limited slack or lag.

In some embodiments, a user may be able to determine whether the infusion process is complete by visually inspecting the position and/or movement of the loading actuator 90. If the loading actuator 90 is positioned close to the barrel 62, the infusion process may be nearly or substantially completed, as the fluid within the barrel 62 may have been expelled out through the outlet port 63 and into the infusion site 88. In some embodiments, the user may be able to visually inspect the syringe to verify the remaining fluid volume and determine whether or not the infusion process is complete.

Once the infusion process is determined to be complete, the user may lock the directional lock 40 to prevent further flow into or out of the syringe 60. The user may then remove the needleset 85 from the infusion site 88 and bubble trap 70. In order to decrease the force required to operate the loading actuator 90, the user may adjust the rate actuator to be at its lowest possible setting. While the loading actuator 90 may be always biased by the energy storing assembly 20, this bias may be reduced as a first end 22A of the spring 22 is moved closer to the anchor point 95. In some embodiments, this reduction in bias may occur by operation of the rate actuator 30. In some embodiments, a user may lock the loading actuator 90 when not in use. The syringe 60, bubble trap 70, and connector 82 assembly may then be removed from the infusion pump 100 and appropriately disposed of, along with the vial(s) 80 and needleset 85.

It should be appreciated that while the infusion pump 100 of FIG. 5 has both syringe filling capabilities and fluid delivery capabilities, infusion pumps or other devices having just one of these capabilities are also contemplated. For example, an infusion pump may require manual filling of the syringe by slowly operating a loading actuator, but may be automatic with regards to fluid delivery, as described previously. Such infusion pumps may be similar in structure to the infusion pump 100 of FIG. 5, but may not include the energy storing member 54. It should be appreciated that the infusion pump may have any other functionality in addition to automatic syringe filling and/or fluid delivery, as the present disclosure is not so limited.

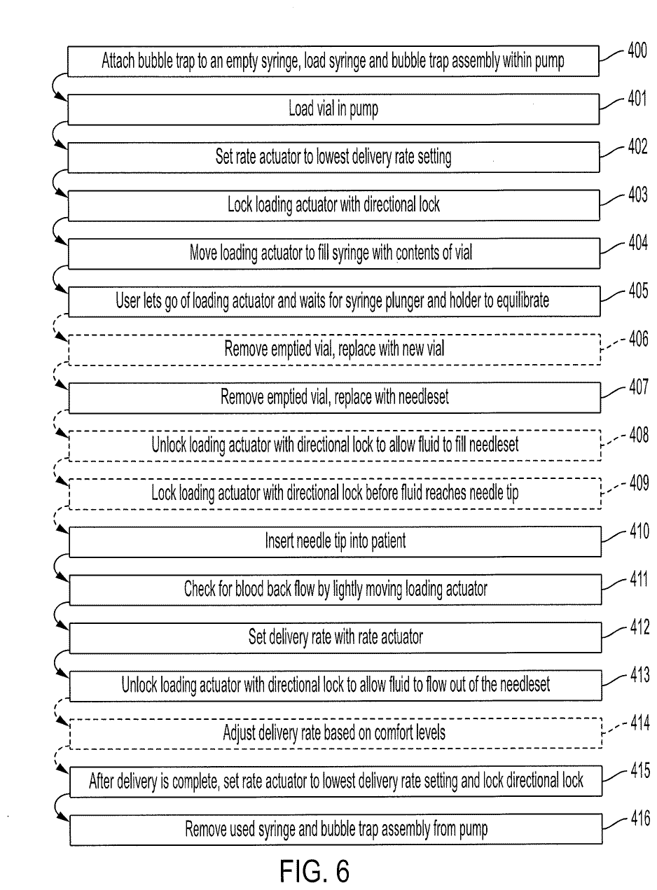

FIG. 6 shows, according to some embodiments, a flow chart for a method of operating an infusion pump to fill a syringe and to deliver fluid. In block 400, a bubble trap is attached to an outlet port of an empty syringe, and the assembly of the bubble trap and syringe are loaded within the infusion pump. In block 401, a vial is loaded into the infusion pump. In some embodiments, loading the vial (which may contain medication) into the pump may fluidically connect the outlet port of the syringe, the bubble trap, and the vial. In block 402, the rate actuator is adjusted to its lowest delivery rate setting. As discussed above, adjusting the rate actuator to its lowest setting may facilitate operation of certain components of the infusion pump (e.g., a loading actuator). In block 403, the loading actuator may then be locked with a directional lock to prevent movement of the loading actuator in at least one direction. The loading actuator may engage with the directional lock in any suitable manner to directionally limit movement of the loading actuator. In block 404, the loading actuator may be operated in order to fill the syringe with fluid from the vial. As described in greater detail above, movement of the loading actuator may result in linear movement of a plunger of the syringe in a proximal direction through a barrel of the syringe, causing fluid to flow from the vial into the syringe barrel. In block 405, the user may release the loading actuator and wait for a desired volume of fluid to flow from the vial into the syringe. It should be appreciated that in embodiments where the directional lock is engaged with the loading actuator, the loading actuator may remain stationary after being released, despite being biased in an opposing direction. Accordingly, the user may not be required to operate the infusion pump in block 405. In block 406, the user may remove the emptied vial from the infusion pump and optionally replace with a new vial. The process of blocks 404-406 may be repeated any number of times to fill the syringe with a desired volume of fluid, as discussed previously. As shown in block 407, the user may remove the emptied vial(s) and instead attach a needleset to the bubble trap. In some embodiments, the user may then choose to optionally prime the needleset by unlocking the loading actuator with the directional lock to allow fluid to fill the needleset, as shown in block 408, and subsequently lock the loading actuator with the directional lock before fluid reaches the needle tip of the needleset, as shown in block 409. The user may subsequently insert the needleset into a desired infusion site on the patient, as shown in block 410. In block 411, the position of the needleset on the patient/user may optionally be verified by checking for blood backflow. In some embodiments, this step may take place by the user first priming the needleset as described above and then reversing fluid flow within the needleset to visually inspect for blood. In block 412, the delivery rate (e.g., fluid flow rate from the syringe) may be adjusted by manipulation of a rate actuator. In block 413, the directional lock may be released to allow fluid to flow out of the needleset and into the patient. It should be appreciated that the directional lock may be engaged at any point during the infusion process to stop fluid flow into the patient. In some embodiments, as depicted in block 414, the user may optionally adjust the delivery rate (e.g., fluid flow rate) to increase comfort or for any other suitable purpose. The delivery rate may be adjusted by a rate actuator, as described previously. In block 415, the user may once again adjust the rate actuator at the lowest delivery rate setting and lock the directional lock. In block 416, the user may remove disposable items from the infusion pump, including the used syringe, vial(s), bubble trap, needleset, and connector, as described previously.

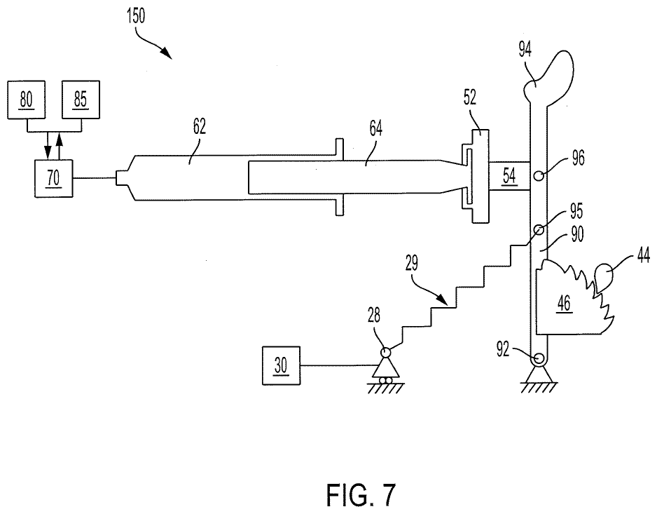

FIG. 7 shows yet another embodiment of an infusion pump 150, which may be similar to infusion pump 100 of FIG. 5, except that the spring 22, roller 26, and connector wire 24 of pump 100 may be replaced by a zero-length spring 29 and a roller joint 28. The zero-length spring 29 may be connected to the roller joint 28 at one end and to an anchor point 95 on a loading actuator 90. The loading actuator 90 may be similar in function to the loading actuator of FIG. 5, such that it may be rotatable around a pinned pivot point 92 at one end and may include a user-operable handle 94 at an opposing end. The roller joint 28 may be able to axially translate in response to operation of a rate actuator 30. In some embodiments, the zero-length spring 29 may have a preload equal to the product of its spring rate and its closed length. The use of a zero-length spring 29 in infusion pump 150 may reduce the overall footprint size and/or weight of the infusion pump compared to that shown in FIG. 5. Of course, while a zero-length spring 29 is shown in FIG. 7, any other means of delivering an adjustable constant force to a plunger holder 52 may also be employed. For example, in some embodiments, the infusion pump may employ a constant force mechanism (e.g., a LaCoste suspension or any equivalent system) or a linear spring. It should also be appreciated that in some embodiments, the infusion pump may deliver a non-adjustable but constant force to a holder, resulting in a constant outflow of fluid from the syringe.

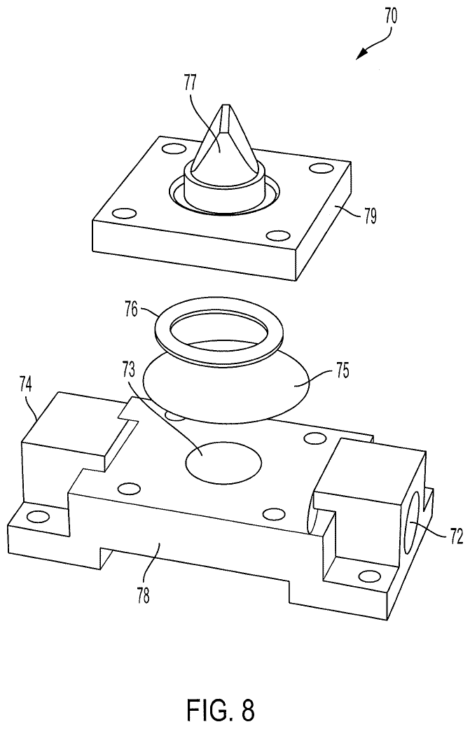

FIG. 8 shows a perspective exploded view of a bubble trap 70 according to some embodiments, which limits the delivery of air bubbles to an infusion site. These air bubbles may be a result of frothing, wherein air bubbles are formed in the syringe when a syringe plunger is quickly pulled away from a syringe barrel. In some embodiments, the air bubbles may be a result of air drawn from an empty vial, or air drawn from a partially filled vial in a tilted configuration (or when the pump is being used when the user is moving, which may cause sloshing in the vial and allow air to flow out of the vial). In some embodiments, the bubble trap may limit the delivery of air bubbles initially located within the needleset tubing, vented spike, syringe, and/or any other component of the infusion pump. Fluid from the syringe and/or vial may have displaced such air bubbles during loading/extraction. In some embodiments, the bubble trap 70 described herein may remove bubbles from the fluid (e.g., medication) before being administered to the infusion site.

The bubble trap 70 may include ports 72, 74 to interface with tubing 65, 83 (see FIG. 5) in order to fluidically connect an outlet port 63 of a syringe 60 and a connector 82. In some embodiments, the ports 72, 74 may be in fluid communication through a channel formed within a body 78 of the bubble trap 70. The channel may include an additional port 73 open to a membrane 75, an o-ring 76, and a check valve 77. The membrane 75 may be a gas-permeable hydrophobic membrane. In some embodiments, the membrane 75 may be a hydrophobic PTFE membrane with a pore size between 50 nm and 100 nm. Of course, other suitable hydrophobic membrane materials and/or sizes are also contemplated. In some embodiments, the bubble trap 70 may be oriented within the infusion pump such that the check valve 77 points away from gravity, as will be discussed in detail further below. In some embodiments, the check valve 77 may be a one-way valve. In some embodiments, the check valve 77 may be a duckbill valve. Of course, other suitable one-way check valves are also contemplated. The bubble trap 70 may also include cap 79 attached to the check valve 77. In some embodiments, the cap 79 may be removably attached to the body 78 to facilitate replacement of the membrane 75 and/or o-ring 76. The cap 79 may be removably attached to the body 78 with any suitable fastening means. In some embodiments, the bubble trap may be a unitary body, such that the user may not have direct access to the membrane 75 and/or o-ring 76.

In operation, fluid flowing through the bubble trap 70 (either from port 73 to port 74 or from port 74 to port 73) may pass over the membrane 75. Given the hydrophobic properties of the membrane 75 as well as the pore size, the membrane 75 may not allow any fluid (e.g., medication) to pass through the port 73 to the check valve 77. During delivery, wherein fluid is passing from the outlet port of a syringe to a needleset, the fluid passing through the bubble trap 70 may be at a positive pressure relative to atmosphere. Accordingly, as fluid flows from the syringe through the bubble trap 70 to the needleset, any air bubbles in the fluid may be driven out through the membrane 75 by the drug fluid pressure. The bubbles may subsequently pass through the check valve 77 to the atmosphere. As noted previously, alignment of the check valve against the direction of gravity may facilitate the flow of air bubbles near the membrane 75 interface, which may then ensure that the bubbles are driven out of the trap 70. During syringe loading and in instances of checking for blood backflow, the fluid within the body 78 may be at a negative pressure relative to atmosphere. In other words, there may be a slight vacuum formed within the fluid connection lines of the infusion pump. Accordingly, the check valve 77 may reduce the likelihood of air being drawn through the membrane 75 into the fluid.

It should be appreciated that in some embodiments, air may be pulled into the syringe during the loading process. However, the bubble trap 70 may help to reduce the amount of air bubbles delivered to the infusion site.

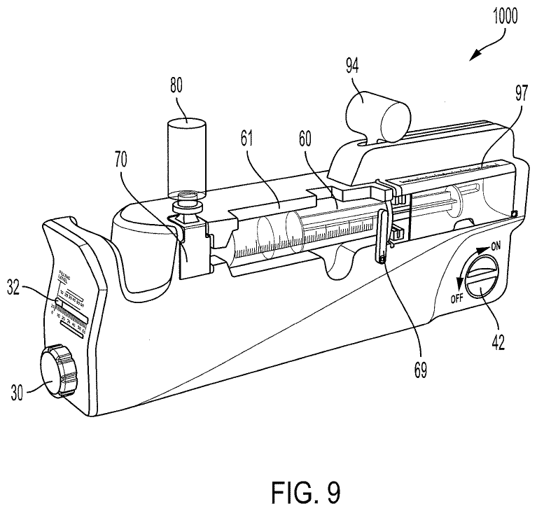

FIGS. 9-11 are various perspective views of yet another infusion pump 1000 according to some embodiments. The pump 1000 may operate functionally similar to the pump 100 shown in FIG. 5, but may include a different arrangement of the components. For example, infusion pump 1000 may include a smooth and elliptical loading handle 94. In some embodiments, the handle 94 may be slightly angled to limit slipping of the infusion pump 1000 during operation. Of course, the handle 94 may have any suitable size/shape to ensure comfort for the user and adequate gripping force. In another example, the infusion pump 1000 may include a rotatable dial as a locking actuator 42. As described above, the locking actuator 42 may be operated to limit movement of a loading handle 94 in at least one direction. In some embodiments, operation of the locking actuator 42 may temporarily fix the loading actuator. The infusion pump 1000 may also include a user-operable rotatable dial as a rate actuator 30.

In some embodiments, the infusion pump 1000 may also include one or more hooks 2 for attachment to straps which may allow the user to carry the infusion pump 1000. In some embodiments, the infusion pump 1000 may be wearable using any combination of features such as straps, buckles, snaps, or any other suitable feature to allow the user to carry the infusion pump 1000. In some embodiments, the infusion pump 1000 may be portable. In some embodiments, the infusion pump 1000 may be portable during fluid (e.g., medication) delivery.

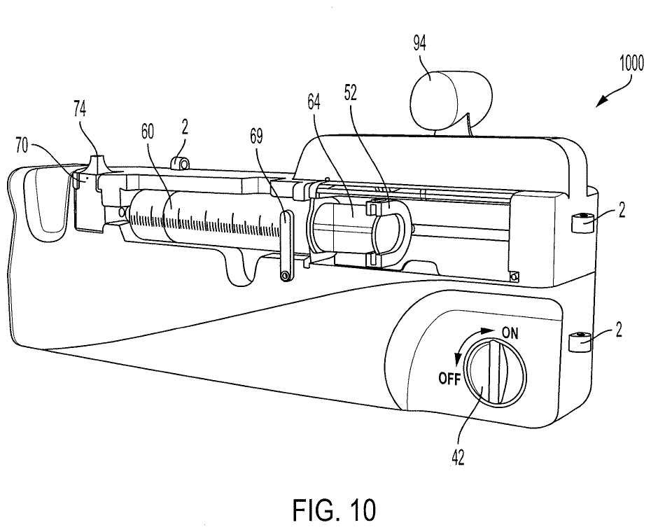

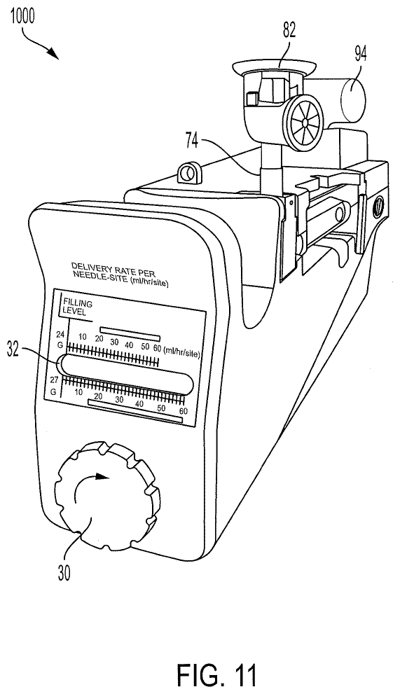

As shown in FIGS. 9 and 10, in some embodiments, one or more vials 80 may be attached to a port 74 of a bubble trap 70 with a spike connector. For example, a vented spike may be installed on each vial, allowing fluid to flow in and out of the vial. The spike and vial assembly may then be installed on a port 74. It should be appreciated that in some embodiments, the vented spike and vial assembly may be disposed of together once sufficient fluid has been extracted from the vial into the syringe. Any suitable vented spike system known in the art may be employed, as the present disclosure is not so limited. In some embodiments, after the syringe is sufficiently filled, the spike and vial assembly may be disconnected from the port 74, and a needleset may then be installed on the port 74 in the same position on the port 74 that the spike was originally connected to, thus replacing the spike and vial assembly. Accordingly, the port 74 may be capable of being interchangeably coupled to a needleset and a vented spike/vial assembly. In some embodiments, one or more vials 80 may be attached to the bubble trap 70 through a connector 82, as shown in FIG. 11. The connector 82 may be disposable. In some embodiments, the connector may include Luer lock connectors to facilitate fluid-tight flow into and out of the bubble trap 70 (and subsequently the syringe 60).

In operation, the syringe 60 may be inserted into a cutout 61 of the infusion pump 1000, as shown in FIG. 9. The cutout 61 may fix the barrel of the syringe 60 in place, while allowing a plunger 64 to be axially translated via a holder 52 (see FIG. 10). In some embodiments, the infusion pump 1000 may include a rotational clip 69 to help hold the syringe 60 in place.

It should be appreciated that the cutout 61 may allow the user to visually inspect the syringe 60. Similarly, the infusion pump 1000 may include a loading indicator 97, which may allow the user to operate the handle 94 to a suitable position corresponding to a desired dosage amount. For example, the loading indicator 97 may include markers indicating various volumes corresponding to the filling volume of the syringe 60. It should be appreciated that the loading indicator may be readily visible to the user during syringe loading. Accordingly, in some embodiments, the loading indicator 97 may be located on the top surface of the pump 1000. The infusion pump 1000 may also include a rate scale 32, as shown in FIGS. 9 and 11. The rate scale 32 may include markers indicating various possible flow rates in operation of the infusion pump 1000. Accordingly, a user may rotate the rate actuator 30 and watch an indicator move on the rate scale 32 to visualize and appropriately select a fluid outflow rate.

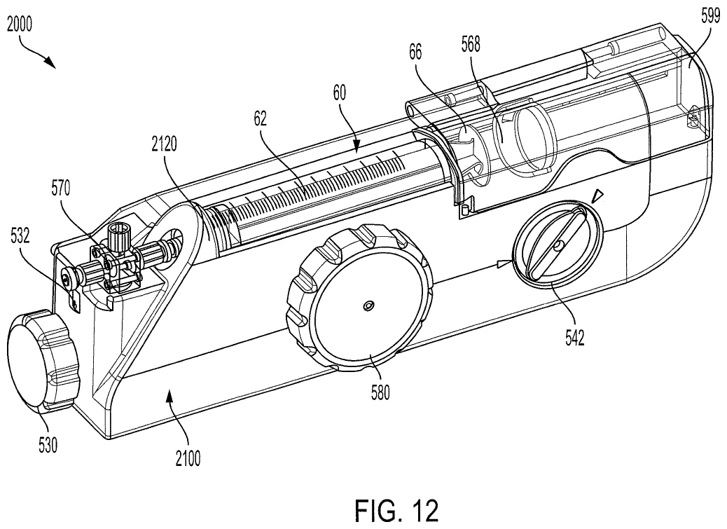

FIGS. 12-21 are various views of yet another infusion pump 2000. FIG. 12 shows the infusion pump 2000 with a housing 2100. The infusion pump 2000 includes a rate actuator 530 for adjusting the rate of fluid delivery out of a needleset which may be connected to an adaptor 570. In some embodiments, a plunger holder 568 may serve to apply pressure to a syringe plunger flange 66 to urge fluid out of a syringe barrel 62 loaded into the pump and through the needleset towards an infusion site (e.g., on a patient). As will be described in greater detail below, the plunger holder 568 may be driven by an internal mechanism which may control the fluid flow rate based on a setting of the rate actuator 530. In some embodiments, the infusion pump 2000 may also include a rate indicator 532 which may display the setting set by the rate actuator 530. A user may therefore adjust the rate actuator 530 to achieve a desirable setting by reading the rate indicator 532. It should be appreciated that although a mechanical rate indicator 532 is depicted in FIGS. 12-21, digital systems may also be employed, as the present disclosure is not so limited.

As shown in FIG. 12, the infusion pump 2000 may also include a lock actuator 542 for locking or unlocking the pump. The lock actuator may serve to inhibit movement of the plunger holder 568, thereby substantially reducing fluid flow into or out of the syringe barrel 62. In some embodiments, as shown in FIG. 12, the lock actuator 542 may be configured as a rotatable thumb turn, movable between a first position to indicate that the pump is “locked” and therefore “off.” and a second position to indicate an “unlocked” pump which is “on”. Of course, other intermediate configurations of the lock actuator 542 are also contemplated, as the present disclosure is not limited by the operation of the lock actuator.

In some embodiments, the infusion pump 2000 may include a hinged cover 599 over the syringe plunger during operation. The cover 599 may be formed of a translucent or transparent material to allow the user to observe the movement of the plunger with reduced risk of accidentally interfering with its movement. In some embodiments, the cover 599 may be closed with the aid of magnets in housing 2100. The user may be able to open the hinged cover 599 to place or remove the syringe from the infusion pump.

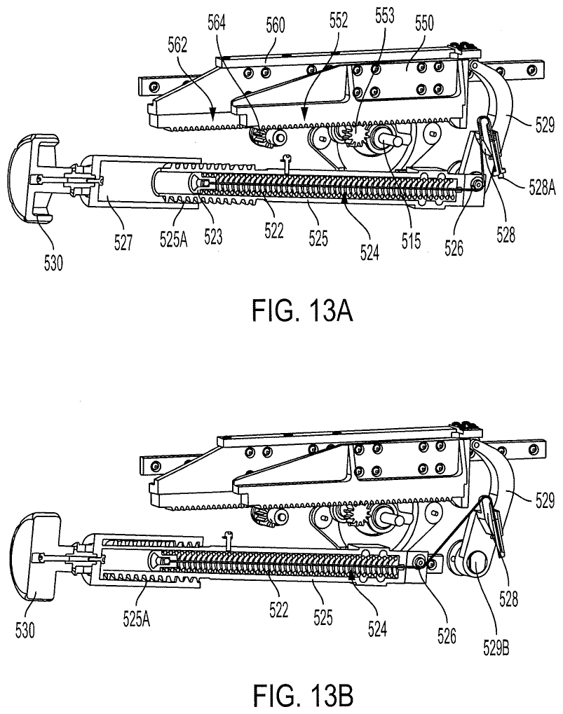

FIGS. 13-21 show various portions of the interior of infusion pump 2000, with housing 2100 removed, according to some embodiments. Infusion pump 2000 operates similarly to infusion pumps described previously in FIGS. 5 and 9, in that it may deliver large volumes of fluid (e.g., medicament) at a constant but adjustable rate. The rate of delivery may be adjusted with rate actuator 530 at any time during delivery to suit the needs of the user (e.g., for comfort).

In some embodiments, the rate actuator 530 may be coupled to a delivery spring 522. As discussed previously in relation to other embodiments of the infusion pump, the spring 522 may be tensioned to adjust the delivery rate of fluid out of the pump. In embodiments captured by FIGS. 12-21, the delivery spring 522 may be housed inside of a delivery sledge 525, as shown in FIGS. 13A-13B. The sledge 525 may include threads 525A which may interact with an internal surface of a nut 527, which may be rotationally coupled to the rate actuator 530. Accordingly, a user may axially displace the delivery sledge 525 relative to the nut 527 with rotation of the rate actuator 530.

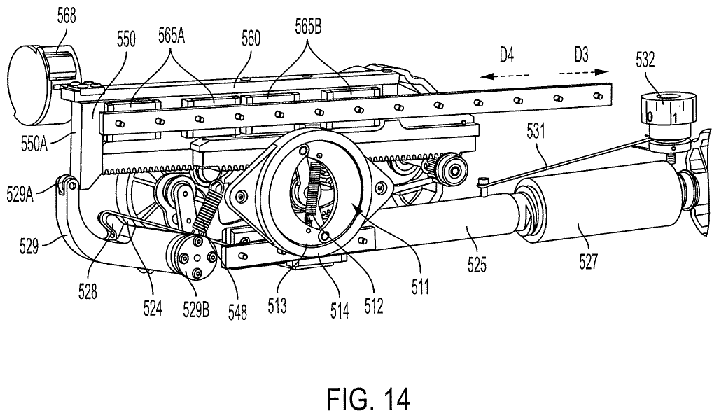

In some embodiments, the delivery spring 522 may be coupled to a connector wire 524, shown in FIGS. 13A-13B. The connector wire 524, which may be configured as a substantially non-extensible cable, may be routed from a connector 523 with the delivery spring 522, through the central hollow portion of the spring 522, around a roller 526 and a projection 528 formed on a drive arm 529, as shown in FIG. 14. In some embodiments, roller 526 may be attached or otherwise coupled to delivery sledge 525 such that it may be displaced along with the sledge 525 along with operation of the rate actuator 530. The connector wire 524 may be anchored at a fixed anchor point 528A on the drive arm 529. The projection 528 may be configured such that tension on the wire 524 may serve to rotate the drive arm 529. In turn, the drive arm may apply a force along direction D3, shown in FIG. 14, to a surface 550A of a driving sledge 550. As will be described in greater detail below, movement of the driving sledge 550 along direction D3 may urge fluid flow out of the infusion pump. In some embodiments, the drive arm 529 may apply a force to the surface 550A of the driving sledge 550 with a roller 529A. Of course, alternative embodiments of force transfer between the drive arm and driving sledge are also contemplated. It should be appreciated that the tensile stiffness of the connector wire 524 may be sufficiently greater than the stiffness of the spring 522, such that the length of the connector wire 524 may remain constant during operation of the infusion pump 2000. In other words, the connector wire 524 may not deform with movement of the delivery sledge 525.

As shown in FIGS. 13A-13B, the delivery spring 522 may be configured as a compression spring in contrast to the tension spring described in relation to FIGS. 5 and 9. Accordingly, axial movement of the delivery sledge 525 towards the nut 527 upon rotation of the rate actuator may serve to compress the delivery spring 522 and tension the connector wire 524. FIG. 13A depicts a delivery spring 522 at a minimum or low delivery rate, whereas FIG. 13B depicts the delivery spring 522 at a maximum or high delivery rate. The compression of the delivery spring 522 may therefore tension the connector wire 524, which is connected to a distal end of the spring 522 at connector 523. It should be appreciated that the connector 523 may be axially moveable relative to the delivery sledge 525 such that the spring may be compressed by the connector wire 524 when the rate actuator 530 is rotated to increase the delivery rate. In some embodiments, employment of a compression spring may reduce the spatial footprint of the delivery system in comparison to a tension spring.

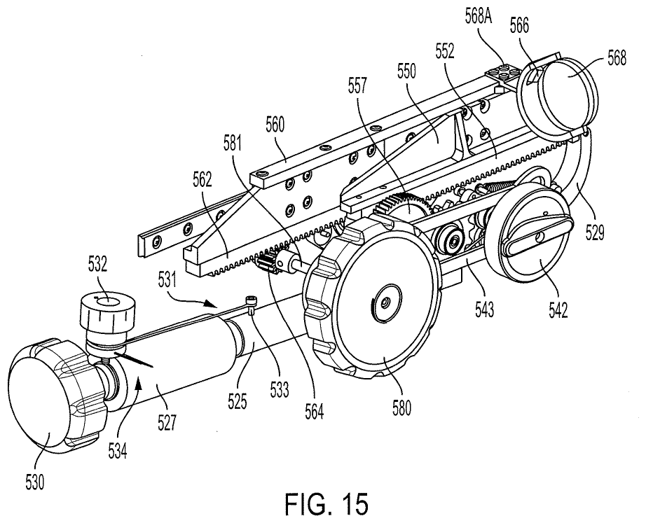

As noted previously, in some embodiments, an infusion pump 2000 may include a rate indicator 532 to communicate the delivery rate setting to the user. In some embodiments, the rate indicator may be operated mechanically. For example, as shown in FIG. 15, the rate indicator may be a rotatable barrel with indicia such as numbers (e.g., indicating the flow rate). The rate indicator 532 may be connected to a cable 531 fixed at one end to a post 533 on the delivery sledge 525, as shown in FIGS. 14-15. The cable 531 may be connected to and wrapped around the indicator 532 such that the indicator may rotate with axial displacement of the sledge 525. As described previously, the delivery rate may be adjusted by the rate actuator 530 axially displacing a delivery spring housed within the delivery sledge. Therefore, the indicator 532 may indicate a change in the delivery rate associated with movement of the delivery sledge. In some embodiments, the indicator may be biased to its maximum value with a torsion spring 534. It should be appreciated that alternative embodiments of the rate indicator may be employed, as the present disclosure is not limited by the function of the rate indicator described herein.

In operation, a user may first load a syringe (see syringe 60 in FIG. 12) into the pump 2000, connect an adapter 570 to said syringe, and load a vial of fluid (e.g., medicament) onto the adapter 570. As will be described in greater detail below, the installation of a vial of fluid on the adapter 570 may place the vial in fluid communication with the syringe barrel 62. The user may then fill the syringe with fluid from the vial without significant force or dexterity. This easy loading of the syringe is facilitated by coupling the syringe plunger to the infusion pump 2000 and operating the plunger with the internal mechanisms of the pump.

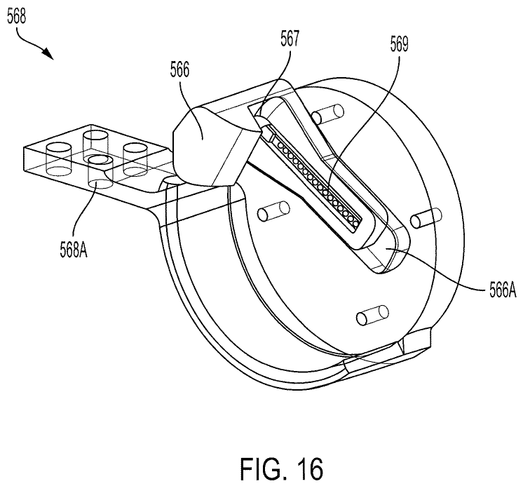

To fill a syringe with fluid from a vial, a user may first couple the syringe plunger to the pump via the plunger holder 568. In operation, the user may position the empty syringe in the pump, abutting the barrel 62 portion of the syringe against surface 2120 (see FIG. 12) of the pump 2000. In some embodiments, the syringe may be positioned in the pump by abutting the syringe plunger flange 67 against the pump housing. It should be appreciated that any arrangement of retaining the syringe within the pump may be employed, as the present disclosure is not so limited. The user may then operate a loading actuator 580 to move the plunger holder close to the syringe plunger flange 66. The plunger holder 568 may include a latch 566, as shown in FIG. 16. The latch 566 may include a curved surface, which may abut against the plunger flange 66. The curved surface may be urged radially outward upon contact with the flange 66. If the user continues to move the holder 568 towards the plunger, the flange 66 may snap into a notch 567 (see FIG. 16) of the plunger holder 568. In this way, the plunger holder 568, axially operable with a loading actuator, may be coupled to the syringe plunger in order to axially move the plunger with limited backlash. In some embodiments, the latch 566 may be radially moveable along a channel 566A in the holder body. The latch 566 may be biased to its lowest radial position in the holder with a spring 569, such that it may retain the plunger flange during operation.

To linearly actuate the plunger holder 568, and subsequently the syringe plunger, the user may operate the loading actuator 580 shown in FIG. 15. The plunger holder 568 may be linearly movable with rotation of the loading actuator 580 in the following way: In some embodiments, the plunger holder 568 may be fixed to a loading sledge 560 via one or more connections 568A, as shown in FIGS. 15-16. In other embodiments, the plunger holder 568 may be formed as part of the loading sledge 560. It should be appreciated that any arrangement between the plunger holder 568 and the loading sledge 560 which linearly fixes the holder and sledge may be employed, as the present disclosure is not so limited. The loading sledge 560 may include a rack 562, which may be engaged with a pinion 564 positioned on a shaft 581 of the loading actuator 580. In this way, as a user rotates the loading actuator 580, the loading sledge 560 and the plunger holder 568 may be linearly actuated. In some embodiments, clockwise rotation of the actuator 580 may result in the plunger holder 568 pulling the syringe plunger away from the syringe barrel (fixed in place), and counterclockwise rotation of the actuator may result in the plunger holder 568 pushing the syringe plunger towards the barrel. Of course, alternative embodiments for linear actuation of the plunger relative to the barrel are also contemplated.

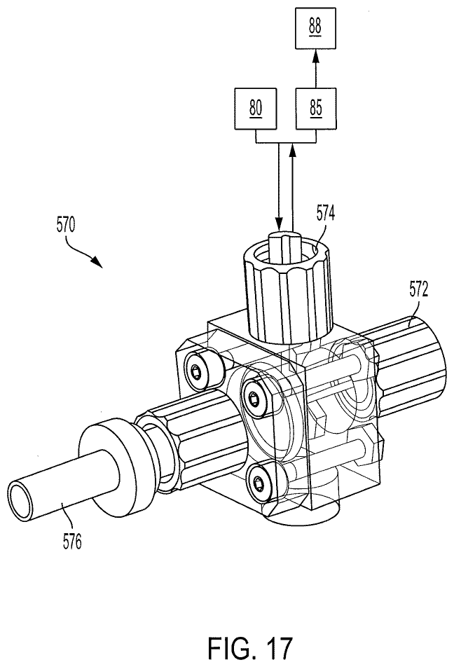

Once a syringe has been latched into infusion pump 2000 via the plunger holder 568, the user can fill the syringe with fluid (e.g., medicament) from a vial. The vial 80 may be installed on adapter 570, shown in FIGS. 12 and 17. In some embodiments, a user may fit a female-to-female Luer lock adaptor to the end of the syringe, which can be engaged with the adapter 570 via a rotating-snap-ring type Luer lock fitting 572. This rotating snap-ring enables the right-angle adaptor to be correctly oriented relative to the pump. In some embodiments, the housing 2100 of the pump may include one or more alignment features to ensure the correct positioning of the adapter 570. In some embodiments, as shown in FIG. 17, the adaptor 570 may be configured as a right-angle adapter. The user may then connect a vial 80 to the adapter 570 at connection or port 574 using a standard piercing connector to place the vial 80 in fluid communication with the syringe. In some embodiments, the adaptor 570 may include a bubble trap 576 to reduce the risk of bubbles flowing from the vial to the syringe, as described previously.

Subsequently, the user may rotate the loading actuator 580 to linearly displace the syringe plunger 66 relative to the barrel, filling the barrel with fluid from the vial 80. It should be appreciated that at this stage of the filling process, the locking actuator 542 is configured in its “off” position, such that movement of the loading sledge 560 is controlled substantially by the loading actuator 580. Of course, movement of the syringe may also be partially limited by the hydraulic resistance of fluid from the vial into the syringe through the adapter.

In some embodiments, the capacity of the syringe barrel may be greater than the volume of the vial. Therefore, more than one vial may be used to fill a given syringe. Once a first vial is depleted into the syringe, the user may remove the vial from the adapter 570 and replace it with a new vial, repeating the same filling process (e.g., manipulating the loading actuator to linearly displace the plunger relative to the barrel). This process may be repeated until the syringe is filled with the desired volume of fluid.

To begin the infusion process, the user may remove the last vial 80 from the infusion pump 2000 and place a needleset 85 in port 574 of the adapter 570. The needleset 85 may then be placed on an infusion site 88 (e.g., on a patient). In some embodiments, the user may elect to conduct a blood back flow test to verify that the infusion site 88 is not in a blood vessel. To do so, the user may first manually turn the loading actuator 580 to urge fluid out of the syringe 60 and into the needleset 85. In some embodiments, the anti-clockwise rotation of the loading actuator 580 may result in the syringe plunger 66 moving closer towards the barrel 62, pushing fluid out of the syringe tip. Once the fluid has reached the infusion site, which may be verified visually, the user may then gently turn the loading actuator 580 in the opposite direction (e.g., clockwise) to urge generate suction in the syringe. The user may then monitor the needleset line to check for signs of blood, as described previously.

Once the user has verified that the infusion site 88 is not positioned in a blood vessel, the infusion process may begin. In embodiments depicted by FIGS. 12-21, the syringe plunger may be linearly actuated at a constant but adjustable rate with the force retained within the delivery spring 522. As described previously, the length of compression delivery spring 522 may be adjusted using the rate actuator. The further the delivery sledge 525 is from the drive arm 529, the greater the distance between the anchor 528A and the roller 526, and therefore the greater the extension of spring 15, and therefore the greater the force exerted by spring 15. Moving the delivery sledge 525 away from pivot point 529B (see FIG. 13B) causes the line of action of the wire 524 to be further from the pivot 529B of the drive arm 529, and therefore the force of the delivery spring 522 transmitted through wire 524 applies a greater torque on the drive arm 529 about its pivot 529B. Accordingly, moving the delivery sledge 525 further from the drive arm pivot 529B increases the force that can be exerted on the syringe, and therefore increases the delivery pressure, thereby increasing the fluid delivery rate.

The force within the delivery spring 522 may be transferred to connecting wire 524, which is wrapped around a projection 528 on a drive arm. The tension within the wire 524 may serve to rotate the drive arm 529 (which is fixed relative to the projection 528) in an anti-clockwise direction (in FIGS. 13A-13B). A roller 529A of the drive arm 529 may apply a force to a surface 550A of the driving sledge 550, as shown in FIG. 14 to drive the syringe plunger toward the syringe barrel and urge fluid out of the syringe.

As described previously relative to infusion pumps of FIGS. 5 and 9, the combination of the connection wire, roller, and spring results in a substantially constant force (and therefore, substantially constant fluid flow rate) applied to the syringe, which can be varied based on the setting of the rate actuator. During the infusion process, the delivery spring 522 is releasing its stored energy by extending back to its original length. This energy is released non-linearly according to Hooke's Law. However, the rotation of the drive arm 529 extends the line of action from the pivot point 529B, such that the connecting wire 524 is extended at a greater distance from the point 529B. Therefore, the combination of linearly decreasing extension force of the delivery spring 522 and the linearly increasing torque arm result in a substantially constant delivery force delivered to the syringe.

In some embodiments, the infusion pump may include a damper 511 configured to mitigate the risk of the delivery sledge 550 moving the syringe plunger too quickly towards the syringe barrel. The damper 511, shown in FIG. 14, may include two weights 513 lightly coupled to one another with spring 512. Under the centrifugal forces of high rotational speed, weights 513 are driven outward, and drag on a circular housing 514. This drag may slow the motion of a ratchet shaft 515 (see FIG. 13A) to a safe speed. It should be appreciated that regular delivery speeds may be too slow to engage the damper, and that the weight may not be in contact with the housing during typical delivery.

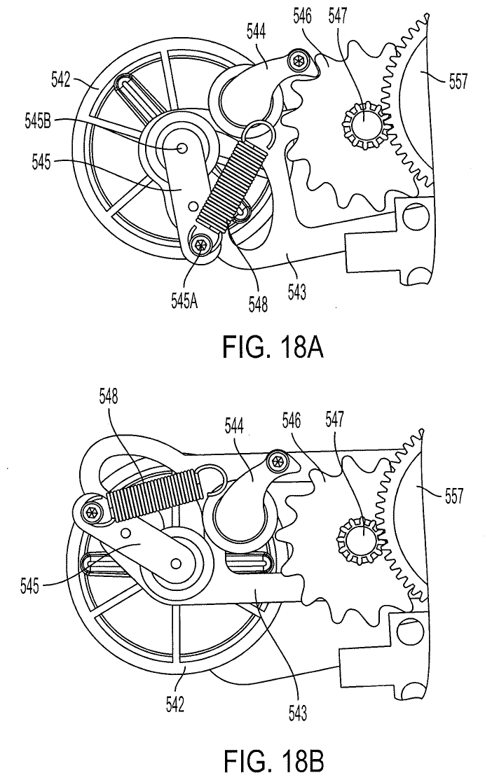

As will be described in detail below, in some embodiments, the ratchet shaft 515 may be coupled to the driving sledge 550 through rack 552 and pinion 553 (see FIGS. 13A-13B). The pinion 553 may be rotationally locked and coaxial with a gear 557 (see FIGS. 18A-18B) engaged with a smaller gear 547 which may be rotationally locked and coaxial with the ratchet 546. The gearing arrangement may serve as a fast-moving, low-torque damper against the forces applied to the driving sledge from the driving arm 529.

To begin infusion, a user may first need to unlock the pump 2000 by manipulating a locking actuator 542 to its “on” position. The actuator may serve to unlock the pump and allow the fluid to be delivered to the infusion site without the user having to manually manipulate the loading actuator 580.

As shown in FIGS. 18A-18B, the locking actuator 542 is rotationally coupled to a locking arm 545, which is engaged with a locking spring 548 at one end 545A. The spring 548 is connected to a pawl 544 at its opposing end, such that it stretches between the locking arm 545 and the pawl 544. FIG. 18A shows the locking actuator 542 in its “off” position. In this position, the pawl 544 is engaged with a ratchet wheel 546 to inhibit the rotation of the wheel 546 in one direction. For example, in FIG. 18A, the ratchet and pawl geometry inhibit the ratchet wheel 546 from moving in the anti-clockwise direction. The ratchet wheel 546 is coaxially fixed to a gear 547, which is configured to engage with a gear 557. In some embodiments, gear 557 may be engaged with the drive sledge 550. For example, as shown in FIGS. 13A-13B, 15, and 19A-19B, the gear 557 may be loaded on the same shaft as pinion 553, such that rotation (or lack thereof) of the gear 557 may result in commensurate rotation of the pinion 553. The pinion 553 is configured to engage with rack 552 of the driving sledge 550 (see FIG. 13A).

Therefore, if the locking actuator 542 is in the “off” position, meaning that the pump is locked, the locking arm 545 may be in the position depicted in FIG. 18A, such that the pawl 544 is engaged with ratchet wheel 546. This engagement inhibits the rotation of the wheel 546 in the clockwise direction. As the ratchet wheel 546 is locked from rotating in one direction, its coaxial gear 547 is also rotationally locked in one direction, as well as gear 557, which is engaged with gear 547, and pinion 553, which is on the same shaft as gear 557. In other words, when the locking actuator 542 is in the “off” position, the driving sledge 550 can only move in one direction (e.g., direction D4 shown in FIG. 14). In this way, the ratchet and pawl engagement act against the force applied to the sledge 550 by the drive arm 529 (from the compression spring 522) to inhibit movement of the sledge in direction D3. This lock reduces the risk of the syringe plunger 66 being pushed towards the barrel prior to infusion (e.g., during syringe filling).

When the user turns the locking actuator 542 to the “on” position to begin infusion, the locking arm 545 may rotate along with the actuator 542, bringing the pawl 544 out of engagement with the ratchet wheel 546, as shown in FIG. 18B. Accordingly, gear 547, gear 557, and pinion 553 are free to rotate in any direction and do not block the driving sledge 550 from moving in direction D3. In this orientation, the drive arm 529 may begin to apply a constant force to the driving sledge surface 550A. In some embodiments, the driving sledge 550 and loading sledge 560 (and accordingly, plunger holder 568) may be coupled in a manner that allows the driving sledge 550 to move the loading sledge 560 in direction D3 (see FIG. 14) but not in direction D4. In some embodiments, the loading sledge 560 and driving sledge 550 may include one or more surface against which they may abut to permit the driving sledge 550 to drive the loading sledge 560 in direction D3 but not direction D4. In other embodiments, an infusion pump may include one or more driving sledge carriages 565A and one or more loading sledge carriages 565B, as shown in FIG. 14. As shown in the figure, the carriages may abut against one another to permit driving of the loading sledge 550 in direction D3 but not in direction D4. As such, when the locking actuator 542 is in the “on” position, the plunger holder 568 may push the syringe plunger with a constant force to urge fluid out at a constant flow rate.

In some embodiments, the fluid delivery rate may be continuously adjusted (e.g., for comfort or logistical reasons) during delivery (e.g., infusion) by manipulating the rate actuator 530. For example, the user may rotate the rate actuator 530 in a clockwise direction to increase the delivery rate or in an anti-clockwise direction to decrease the delivery rate. It should be appreciated that fluid delivery need not be stopped during this process.

It should be appreciated that the inclusion of two independent sledges (loading sledge 560 and driving sledge 550) allows the user to move the plunger holder 568 (fixed to the loading sledge 560) without having to overcome the force applied by the drive arm 529 on the driving sledge 550 when the pump is locked. In other words, a user may linearly displace the syringe plunger without significant dexterity or force, when the pump is locked. Although embodiments with a single sledge capable of both loading and driving are also contemplated, wherein the ratchet lock acts directly on the drive arm, the large torques exerted on the drive arm may require larger gears than the two-sledge configuration, which may increase the overall footprint of the pump.

In some embodiments, the gear ratio between gear 557 and gear 547 may be 5:1 to maximize the resolution of the lock. In other words, fluid delivery may be stopped much sooner after locking the actuator (and engaging the pawl 544 with the ratchet wheel 546) than if the gear was larger, as the time to stop is dependent on the size of the teeth of the gear 547. It should be appreciated that the gear ratio between gear 557 and gear 547 may be any suitable ratio, including, but not limited to, at least 1:1, 2:1, 3:1, 4:1, 5:1, 7:1, 10:1, 20:1, and/or any other suitable ratio. The gear ratio between gear 557 and gear 547 may also be less than or equal to 20:1, 10:1, 7:1, 5:1, 4:1, 3:1, 2:1, 1:1, and/or any other suitable ratio. Combinations of the foregoing ranges, including from 1:1 to 20:1 are also contemplated, as the present disclosure is not limited by the gear ratio between gear 557 and gear 547.

It should be appreciated that the function of the loading actuator to linearly actuate the plunger holder via the engagement of the rack of the loading sledge and pinion of the loading actuator may provide a finer control over the linear actuation of the plunger. As noted previously, if the user elects to adjust the delivery rate to its minimum value, the user may not need to be working against the driving spring to operate the loading actuator. These two features enable the user to load fluid from a vial into a syringe with less force and dexterity compared to current state of the art systems. In some embodiments, the infusion systems described herein may be operated one-handedly, which could enhance the usability and accessibility of the pumps.

In some embodiments, unlocking the locking actuator 542 may include a secondary function of decoupling the loading actuator 580 from the loading sledge 550 (and therefore the plunger holder 568) during infusion. This may reduce the risk of the user accidentally interacting with the loading actuator 580 and disrupting the constant flow of fluid to the infusion site. This safety feature may be accomplished using a clutch mechanism.

In some embodiments, turning the locking actuator 542 to the “on” position may also disengage a spring dog clutch between the loading actuator 580 and the pinion 564, located on the same shaft 581 (shown in FIG. 15). Accordingly, manipulation of the loading actuator 580 may not result in any movement of the loading sledge 560, which is connected to the pinion 564 via the rack 562. In other words, the loading actuator 580 may be freely manipulated without affecting fluid delivery when the locking actuator 542 is in the “on” position (e.g., during infusion).

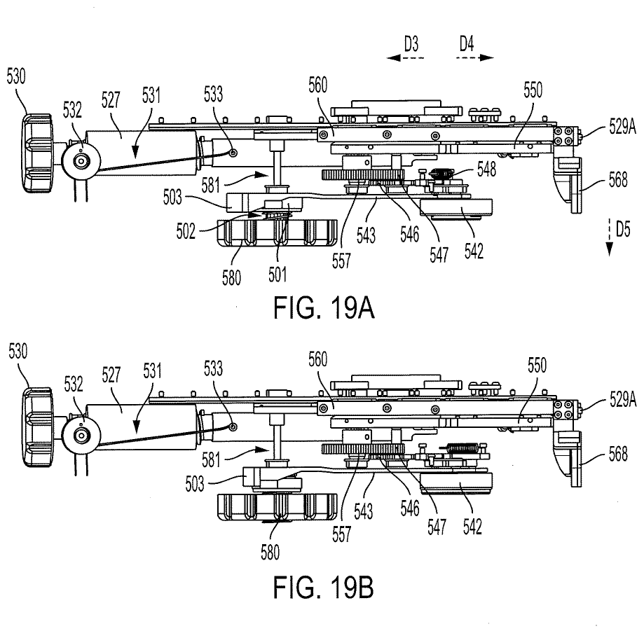

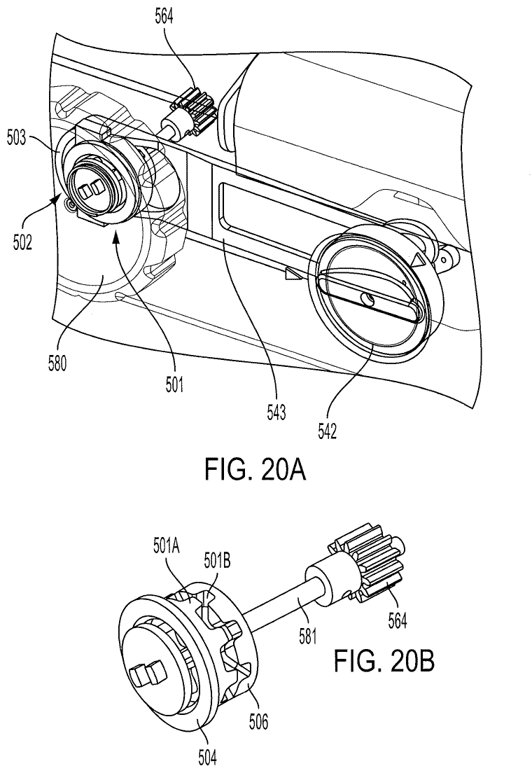

As shown in FIGS. 19A-21B, the clutch mechanism may include a bracket 543, a spring 502, and a dog clutch 501. The dog clutch may include a first portion 504 axially moveable along loading actuator shaft 581. The first portion 504 may include one or more projections 501A (see FIG. 20B) configured to engage with depressions 501B in a second portion 506. In some embodiments, first portion 504 may be rotatably coupled to the loading actuator 580, whereas the second portion 506 may be fixed to shaft 581. Accordingly, disengagement of projections 501A from depressions 501B when turning the lock actuator 542 to the “on” position may serve to decouple the loading actuator from the loading sledge 560. Accordingly, the risk of unintentional interference with the fluid delivery may be minimized.

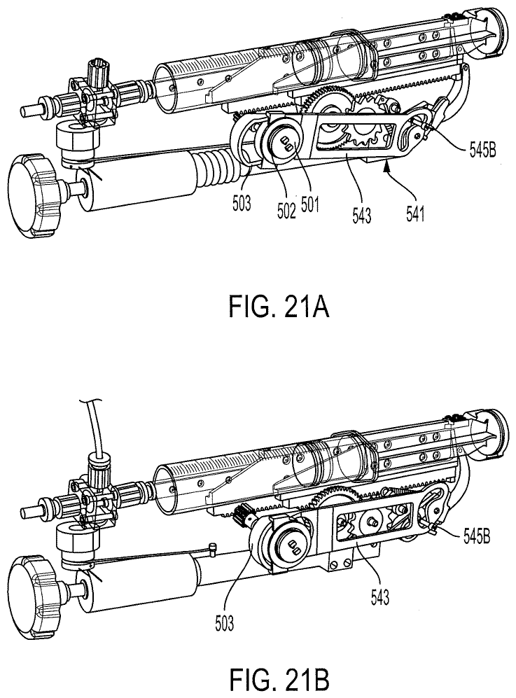

In some embodiments, rotating the locking actuator 542 to the “on” position may urge a pin 545B (see FIGS. 18A-18B and 21A-21B) to translate along a cam slot 541 formed in the bracket 543. This movement may urge the bracket 543, as well as its clamp portion 503, in direction D3 due to the geometry of the cam slot 541. As the clamp portion 503 moves in direction D3, it may abut against a surface of the dog clutch 501, pushing the first portion 504 in direction D5 (see FIG. 19A), which may serve to disengage projections 501A from depressions 501B of the second portion 506. In this way, the operation of the locking actuator 542 to the “on” position may decouple the loading actuator 580 from the loading actuator shaft 581, thereby decoupling the loading actuator 580 from the loading sledge 560. Similarly, rotating the locking actuator 542 to the “off” position may urge the bracket 543 in direction D3, allowing the spring 502 to extend, which may subsequently urge the first portion 504 towards the second portion 506 in order to engage the projections 501A of the first portion 504 into the depressions 501B of the second portion. In this way, the loading actuator 580 may be re-coupled to the loading sledge 560. Of course, alternative embodiments of decoupling the loading actuator and the loading sledge are also contemplated as the present disclosure is not limited by the clutch mechanism described herein.

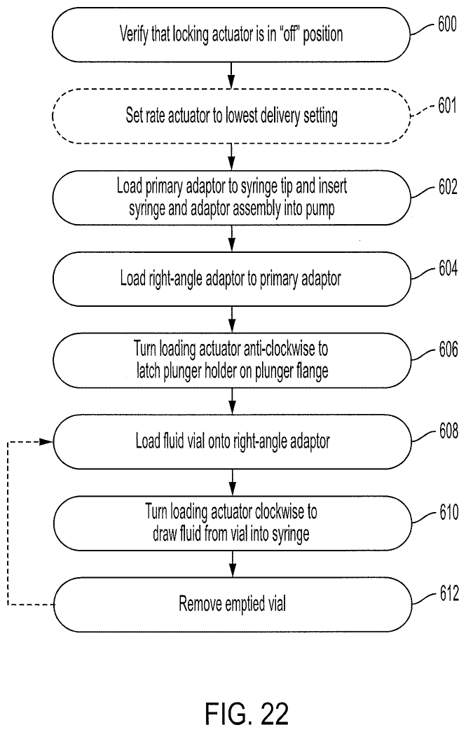

FIG. 22 shows, according to some embodiments, a flow chart for a method of operating an infusion pump to fill a syringe with fluid. In block 600, a user may first verify that the unlocking actuator is in the “off” position to ensure that the energy stored in the delivery spring does not bias a delivery sledge configured to push the syringe's plunger toward its barrel. In block 601, the user may optionally choose to set the rate actuator to the lowest delivery setting to reduce the overall tension within the system and to minimize the force that the delivery sledge applies to the syringe plunger, even though the infusion pump is in its “off” configuration.

In block 602, the user may connect a primary adaptor (e.g., a female-to-female Luer lock adapter) to a syringe and place the assembly of the syringe and primary adapter into the infusion pump. As discussed previously, the infusion pump housing may include one or more features to align and retain the syringe relative to the pump. In block 604, the user may then connect a right-angle adapter to the primary adapter on the syringe. It should be appreciated that although a right-angle adapter is described herein, alternative arrangements of adapters may be employed as the present disclosure is not so limited.

In block 606, the user may then turn the loading actuator anti-clockwise to engage or latch the plunger flange of the syringe with a plunger holder of the pump. As described previously, this engagement will allow the user to push and pull the syringe plunger with a loading sledge operatively connected to the plunger holder. In block 608, the user may load a vial of fluid (e.g., medicament) on to the right-angle adapter, placing the vial in fluid communication with the syringe. In block 610, the user may turn the loading actuator clockwise to draw fluid from the vial into the syringe. Once a desired volume of fluid has been loaded into the syringe, the user may then remove the vial, as depicted in block 612. In some embodiments, a user may optionally elect to load another vial of fluid to further fill the syringe.

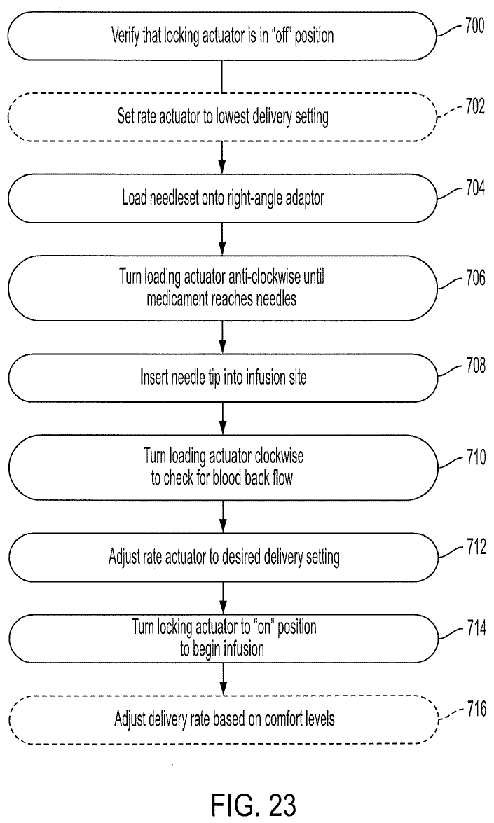

FIG. 23 shows, according to some embodiments, a flow chart for a method of operating an infusion pump to deliver fluid to an infusion site. In block 700, a user may first verify that the unlocking actuator is in the “off” position, and in block 702, the user may optionally elect to set the rate actuator to its lowest delivery settings, as previously described. In block 704, the user may then load a needleset to the right-angle adapter (and/or any other suitable adapter) described in relation to FIG. 22. In block 706, the user may turn the loading actuator anti-clockwise until medicament reaches the needles, which may be located near the infusion site. The user may then insert the needle tip(s) into the infusion site (e.g., the patient), as shown in block 708. In block 710, the user may gently turn the loading actuator clockwise to check for blood back flow within the needleset. If the user detects blood within the needleset, they may elect to adjust the position of the needle at the infusion site. In block 712, the user may manipulate the rate actuator to achieve the desired fluid delivery rate. As described previously, in some embodiments, the user may visualize the delivery rate with a rate indicator, and may subsequently choose to adjust the rate based on comfort and/or medicinal needs. In block 714, the user may turn the locking actuator to the “on” position, unlocking the delivery sledge, as described previously, and allowing a drive arm to apply a continuous and constant force to the syringe plunger. In some embodiments, the user may elect to adjust the fluid delivery rate for comfort or logistical purposes, as shown in optional block 716.

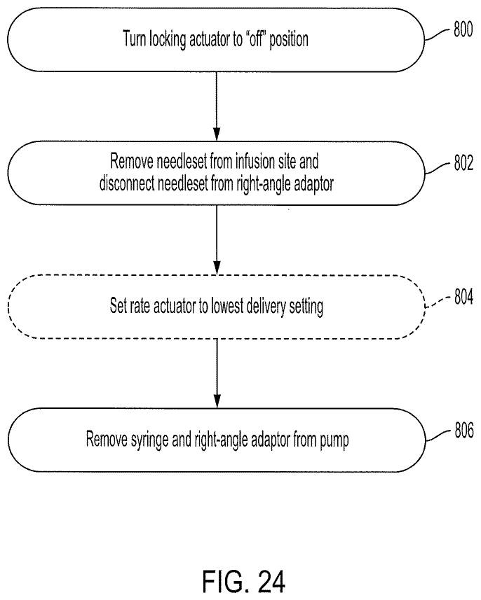

FIG. 24 shows, according to some embodiments, a flow chart for a method of unloading an infusion pump. In block 800, a user may first turn the locking actuator of FIGS. 22-23 to the “off” position to stop the flow of fluid from the syringe to the infusion site. The user may then remove the needleset from the infusion site and disconnect said needleset from the right-angle adapter of FIG. 22, as shown in block 802. In some embodiments, a user may elect to operate the rate actuator to set the fluid delivery rate to its minimum value to reduce the overall tension within the system, as shown in block 804. The user may then remove and dispose of the syringe and right-angle adapter (and/or any other suitable adapter) from the pump, as shown in block 806.

It should be appreciated that infusion pump 2000 shown in FIGS. 12-21 may include any number of features disclosed in relation to infusion pumps 100 and 1000. It should also be appreciated that any infusion pump described herein may include any suitable ergonomic or visually useful features, as the present disclosure is not so limited.

It should be appreciated that while the infusion pumps described herein may be mechanically operated, the pumps may include electrical features for purposes other than syringe loading and/or drug delivery. For example, an infusion pump according to some embodiments may be able to communicate, wirelessly and/or via a wired connection, with one or more external devices, such as a mobile device (e.g., a smartphone, tablet). The mobile device may be running an application that is designed for use with the infusion pump. For example, the infusion pump may include a transmitter, which may collect information from one or more sensors within the infusion pump (e.g., humidity sensors, temperature sensors, timers) and communicate associated information to an external device.

According to one aspect, an infusion pump may directly and/or indirectly interact with a multitude of different parties that may utilize information from the infusion pump. In some embodiments, an infusion pump may communicate directly or indirectly with a healthcare provider, such as a hospital, clinic, and personnel such as a nurse or physician. The healthcare provider may obtain the information from a remote server or other external device such as a mobile device that receives the information from the infusion pump. In some embodiments, the healthcare provider may be in direct communication with the infusion pump. Information that may be sent to a healthcare provider includes, but is not limited to, when an infusion process was completed, much medication was delivered, what the infusion delivery profile was, patient symptoms experienced, and so on. The healthcare provider may use the information to monitor patient adherence and/or to determine efficacy of the medication and/or the dosage regimen of the medication for the patient. In some embodiments, the information communicated from the infusion pump may be integrated with a patient's Electronic Health Record.

In some embodiments, an infusion pump may communicate directly or indirectly with payers, also known as insurance companies. Payers may use information from the infusion pump to monitor aspects such as patient adherence, medication efficacy, and treatment regimen efficacy.

In some embodiments, information relayed through communication from and/or to an infusion pump may be used for data analytics, which may be used by a variety of parties. For example, a supplier (e.g., a producer of a medication and/or an infusion pump) may use information from an infusion pump to determine what features are used most by users, and when (e.g., what buttons/control options are users selecting, on the infusion pump and/or on a companion app on a mobile device), what errors or issues are occurring, and so on. The information may be filterable into different categories such as age, gender, income, experience level, etc.

In some embodiments, information gathered for data analytics may be anonymous and free of PHI (patient health information). In other embodiments, however, the information may contain PHI.

In some embodiments, information gathered from an infusion pump may help provide performance of a medication. The inventors have appreciated that, outside of clinical trials, it can be difficult to assess the performance of a medication when it has been disseminated into the wider public. Communication from infusion pumps and other sources such as mobile devices and/or healthcare providers can help provide information regarding a medication and/or an infusion pump's performance. Information regarding a patient's symptoms and treatment progress may be gathered from patients, e.g., via an electronic symptom diary built into an infusion pump or into a companion app running on a mobile device, and/or may be gathered from a healthcare provider's notes taken during a patient's office visit. The gathered information may help to inform future formulations and/or infusion pump designs for the supplier, and positive performance may be used to help promote use of the medication.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

What is claimed is:

1. An infusion pump comprising:
a holder configured to couple to a syringe plunger and to push and pull the syringe plunger, the holder being moveable between a first position and a second position, and the holder being biased towards the first position;
a loading actuator operatively coupled to the holder, wherein operation of the loading actuator causes the holder to move between the first position and the second position; and
a lock configured to releasably fix the holder in the second position.

2. The infusion pump of claim 1, further comprising an energy storing member configured to bias the holder towards the first position.

3. The infusion pump of claim 1, further comprising a first energy storing member operatively coupled between the holder and the loading actuator.

4. The infusion pump of claim 3, further comprising a second energy storing member configured to bias the holder towards the first position.

5. The infusion pump of claim 1, wherein the lock comprises a ratchet.

6. The infusion pump of claim 1, wherein the lock comprises a pad and brake, the brake configured to frictionally engage with the pad.

7. The infusion pump of claim 1, further comprising a delivery actuator configured to release the lock from fixing the holder in the second position.

8. The infusion pump of claim 7, wherein the delivery actuator is rotatable.

9. The infusion pump of claim 7, wherein the loading actuator is a linearly slidable handle.

10. The infusion pump of claim 3, further comprising a rate actuator configured to adjust a magnitude of a biasing force applied by the first energy storing member.

11. The infusion pump of claim 1, further comprising a bubble trap configured to reduce air bubbles.

12. The infusion pump of claim 11, further comprising:
a first port configured to receive fluid from a vial; and
a second port configured to couple to an outlet end of a syringe,
wherein the bubble trap is fluidly connected between the first port and the second port.

13. The infusion pump of claim 11, wherein the bubble trap comprises a hydrophobic membrane and a one-way valve.

14. The infusion pump of claim 1, further comprising:
a first port configured to receive fluid from a vial; and
a second port configured to couple to an outlet end of a syringe.

15. The infusion pump of claim 14, wherein the first port is configured to removably couple to a spike connector.

16. The infusion pump of claim 14, wherein the first port is configured to removably couple to an infusion set.

17. The infusion pump of claim 1, wherein the holder is moveable to a plurality of positions between the first position and the second position.

18. The infusion pump of claim 17, wherein the lock is configured to releasably fix the holder in each of the plurality of positions.

19. The infusion pump of one of claims 1 to 18, wherein the infusion pump is non-electric.

20. A method of operating an infusion pump, the method comprising:
providing an infusion pump having a holder and a port;
coupling a syringe plunger of a syringe to the holder;
fluidly coupling a vial to the port; and
moving the holder from a first position to a second position to cause the holder to move the syringe plunger in a fill direction and to cause liquid medicament from the vial to flow through the port and enter the syringe.

21. The method of claim 20, wherein moving the holder from the first position to the second position comprises moving the holder against a biasing force that urges the holder towards the first position.

22. The method of claim 20, further comprising locking the holder in the second position.

23. The method of claim 22, further comprising unlocking the holder from the second position to allow the holder to move from the second position to the first position.

24. The method of claim 22, wherein the step of locking the holder in the second position comprises locking the holder in the second position with a ratchet.

25. The method of claim 22, wherein the step of locking the holder in the second position comprises engaging a brake with a pad and generating friction between the brake and the pad.

26. The method of claim 20, wherein the step of moving the holder from the first position to the second position comprises operating a loading actuator.

27. The method of claim 26, wherein the step of operating the loading actuator comprises sliding the loading actuator linearly.

28. The method of claim 26, wherein the step of operating the loading actuator comprises moving the holder against a biasing force that urges the holder towards the first position.

29. The method of any one of claim 21 or 28, further comprising operating a rate actuator to adjust the biasing force.

30. The method of any one of claim 21 or 28, wherein the biasing force is provided by an energy storing member.

31. The method of claim 26, wherein a first energy storing member operatively couples the holder and the loading actuator.

32. The method of claim 31, wherein a second energy storing member biases the holder towards the first position.

33. The method of claim 20, wherein the liquid medicament from the vial flows through a bubble trap prior to entering the syringe.

34. The method of claim 33, wherein the bubble trap comprises a hydrophobic membrane and a one-way valve.

35. The method of any one of claims 20 to 34, wherein the infusion pump is non-electric.

36. An infusion pump comprising:
a holder configured to removably couple to a syringe plunger;
a loading actuator operatively coupled to the holder, the loading actuator being moveable between a first position and a second position;
an energy storing member operatively coupled between the holder and the loading actuator, wherein movement of the loading actuator from the first position to the second position loads the energy storing member; and
a lock configured to releasably fix the loading actuator in the second position.

37. The infusion pump of claim 36, further comprising a slider coupled to the loading actuator to allow the loading actuator to move between the first position and the second position, wherein the slider is configured to slide linearly, wherein the holder is configured to slide linearly, and wherein the energy storing member is coupled between the slider and the holder.

38. The infusion pump of any one of claims 36 and 37, wherein the energy storing member comprises a spring.

39. The infusion pump of claim 36, wherein the lock comprises a ratchet.

40. The infusion pump of claim 36, wherein the lock comprises a pad and brake, the brake configured to frictionally engage with the pad.

41. The infusion pump of claim 36, further comprising a delivery actuator configured to release the lock from fixing the loading actuator in the second position.

42. The infusion pump of claim 36, further comprising a second energy storing member that biases the loading actuator towards the first position.

43. The infusion pump of claim 41, wherein the delivery actuator is rotatable.

44. The infusion pump of claim 36, wherein the loading actuator is a linearly slidable handle.

45. The infusion pump of claim 36, further comprising a bubble trap configured to reduce air bubbles.

46. The infusion pump of claim 45, further comprising:
a first port configured to receive fluid from a vial; and
a second port configured to receive an outlet end of a syringe,
wherein the bubble trap is fluidly connected between the first port and the second port.

47. The infusion pump of claim 45, wherein the bubble trap comprises a hydrophobic membrane and a one-way valve.

48. The infusion pump of claim 36, further comprising:
a first port configured to receive fluid from a vial; and
a second port configured to receive an outlet end of a syringe.

49. The infusion pump of claim 48, wherein the first port is configured to removably couple to a spike connector.

50. The infusion pump of claim 49, wherein the first port is configured to removably couple to an infusion set.

51. The infusion pump of claim 36, wherein the loading actuator is moveable to a plurality of positions between the first position and the second position.

52. The infusion pump of claim 51, wherein the lock is configured to releasably fix the loading actuator in each of the plurality of positions.

53. The infusion pump of one of claims 36 to 52, wherein the infusion pump is non-electric.

54. A method of operating an infusion pump, the method comprising:
providing an infusion pump having a holder;
coupling a syringe plunger of a syringe to the holder;
moving a loading actuator from a first position to a second position, causing loading of an energy storing member that is operatively coupled between the holder and the loading actuator; and
locking the loading actuator in the second position,
wherein the loaded energy storing member exerts a force on the holder, causing the holder to move the syringe plunger in a fill direction, and causing liquid medicament from a vial to enter the syringe.

55. The method of claim 54, further comprising biasing the loading actuator towards the first position by a second energy storing member.

56. The method of claim 55, further comprising operating a rate actuator to adjust a biasing force provided by the second energy storing member.

57. The method of claim 54, wherein locking the loading actuator in the second position comprises engaging a pawl with a portion of a ratchet.

58. The method of claim 54, wherein locking the loading actuator comprises engaging a brake with a pad and generating friction between the brake and the pad.

59. The method of claim 54, further comprising releasing the locked loading actuator by moving a delivery actuator.

60. The method of claim 59, wherein releasing the delivery actuator allows the loading actuator to move towards the first position.

61. The method of claim 59, wherein moving the delivery actuator comprising turning the delivery actuator, the delivery actuator comprising a rotatable knob.

62. The method of claim 54, wherein the step of moving the loading actuator comprises sliding the loading actuator linearly.

63. The method of claim 54, wherein the step of moving the loading actuator comprises rotating the loading actuator.

64. The method of claim 54, wherein the liquid medicament from the vial flows through a bubble trap prior to entering the syringe.

65. The method of claim 64, wherein the bubble trap comprises a hydrophobic membrane and a one-way valve.

66. The method of any one of claims 54 to 65, wherein the infusion pump is non-electric.

67. An infusion pump comprising:
a contact surface configured to contact a syringe plunger, the contact surface being moveable between a first position and a second position;
an energy storing assembly configured to move the contact surface from the second position to the first position by applying a substantially constant force to the contact surface; and
a rate actuator operatively coupled to the energy storing assembly, the rate actuator configured to mechanically adjust a magnitude of the substantially constant force.

68. The infusion pump of claim 67, wherein the energy storing assembly comprises:
an energy storing member operatively coupled to the rate actuator, the energy storing member providing a variable output force; and
a linkage configured to convert the variable output force of the energy storing member into the substantially constant force, the linkage being operatively coupled to the contact surface.

69. The infusion pump of claim 67, wherein the rate actuator is non-electric.

70. The infusion pump of any one of claims 67 and 68, further comprising a loading actuator, wherein operating the loading actuator loads the energy storing assembly and moves the contact surface to the second position.

71. The infusion pump of claim 70, wherein the loading actuator comprises a rotatable lever.

72. The infusion pump of claim 67, wherein the energy storing assembly comprises:
a spring operatively coupled to the rate actuator at one end of the spring;
a cable operatively coupled to an opposing end of the spring; and
a lever attached to the cable at a first portion of the lever, the contact surface being operatively coupled to a second portion of the lever.

73. The infusion pump of claim 72, wherein the energy storing assembly includes a spring having a first end and an opposing second end, the second end being operatively coupled to the contact surface, wherein operating the rate actuator moves the position of the first end relative to the lever.

74. The infusion pump of claim 73, wherein the first end of the spring is fixed to a slider, wherein operating the rate actuator moves the slider relative to the lever.

75. The infusion pump of claim 67, wherein the energy storing assembly includes a constant force spring.

76. The infusion pump of claim 68, wherein the energy storing assembly includes a zero-length spring.

77. The infusion pump of claim 67, wherein the energy storing assembly includes a linear spring.

78. The infusion pump of claim 67, wherein the rate actuator comprises a rotatable knob.

79. The infusion pump of claim 67, further comprising a lock configured to prohibit movement of the contact surface towards the first position.

80. The infusion pump of claim 79, wherein the lock comprises a ratchet.

81. The infusion pump of claim 79, wherein the lock comprises a pad and brake, the brake configured to frictionally engage with the pad.

82. The infusion pump of claim 67, wherein the rate actuator is configured to adjust the magnitude of the substantially constant force between 10 and 200 N.

83. The infusion pump of claim 67, further comprising a bubble trap configured to reduce air bubbles.

84. The infusion pump of claim 83, further comprising:
a first port configured to receive fluid from a vial; and
a second port configured to receive an outlet end of a syringe,
wherein the bubble trap is fluidly connected between the first port and the second port.

85. The infusion pump of claim 83, wherein the bubble trap comprises a hydrophobic membrane and a one-way valve.

86. The infusion pump of claim 67, wherein the contact surface is moveable to a plurality of positions between the first position and the second position.

87. The infusion pump of claim 86, further comprising a lock configured to releasably fix the contact surface in each of the plurality of positions.

88. The infusion pump of one of claims 67 to 87, wherein the infusion pump is non-electric.

89. The infusion pump of claim 68, wherein the energy storing member is one selected from the group of a constant force spring, a zero-length spring, and a linear spring.

90. A method of operating an infusion pump, the method comprising:
operating a loading actuator to load an energy storing assembly, the energy storing assembly becoming locked in a loaded state;
unlocking the energy storing assembly from the loaded state, causing the energy storing assembly to unload and to produce a substantially constant force that is transmitted to a contact surface, causing the contact surface to abut against and move a syringe plunger in a dispensing direction; and
operating a rate actuator to mechanically adjust a magnitude of the substantially constant force.

91. The method of claim 90, wherein the energy storing assembly includes a spring having a first end and an opposing second end, the second end being operatively coupled to the loading actuator, wherein the step of operating the rate actuator comprises moving the position of the first end relative to rate actuator.

92. The method of claim 90, wherein the rate actuator is non-electric.

93. The method of claim 90, wherein the step of operating the rate actuator comprises increasing the magnitude of the substantially constant force by increasing loading of the energy storing assembly.

94. The method of claim 90, wherein the step of operating the rate actuator comprises decreasing the magnitude of the substantially constant force by decreasing loading of the energy storing assembly.

95. The method of claim 90, wherein the energy storing assembly comprises a spring and a linkage, wherein the linkage converts a variable force of the spring into the substantially constant force.

96. The method of claim 95, wherein the spring is operatively coupled to the rate actuator at one end of the spring, and wherein the linkage comprises:
a cable operatively coupled to an opposing end of the spring; and
a lever attached to the cable at a first portion of the lever, the contact surface being operatively coupled to a second portion of the lever.

97. The method of claim 90, wherein the energy storing assembly becomes locked in a loaded state by a ratchet.

98. The method of claim 90, wherein the step of unlocking the energy storing assembly comprises moving a delivery actuator.

99. The method of any one of claims 90 to 98, wherein the infusion pump is non-electric.

100. An infusion pump comprising:
a contact surface configured to contact a syringe plunger, the contact surface being moveable between a first position and a second position; and
an energy storing assembly configured to move the contact surface from the second position to the first position by applying a substantially constant force to the contact surface; the energy storing assembly comprising:
a linkage operatively coupled to the contact surface; and
an energy storing member providing a variable output force,
wherein the linkage is configured to convert the variable output force of the energy storing member into the substantially constant force.

101. The infusion pump of claim 100, further comprising a rate actuator operatively coupled to the energy storing assembly, the rate actuator configured to mechanically adjust a magnitude of the substantially constant force.

102. The infusion pump of claim 101, wherein the rate actuator is non-electric.

103. The infusion pump of any one of claims 100 to 102, further comprising a loading actuator, wherein operating the loading actuator loads the energy storing assembly and moves the contact surface to the second position.

104. The infusion pump of claim 103, wherein the loading actuator comprises a rotatable lever.

105. The infusion pump of claim 101, wherein the energy storing assembly further comprises:
a cable operatively coupled to one end of the energy storing member; and
a lever attached to the cable at a first portion of the lever, the contact surface being operatively coupled to a second portion of the lever, wherein the energy storing member is operatively coupled to the rate actuator at an opposing end.

106. The infusion pump of claim 100, further comprising a lock configured to prohibit movement of the contact surface towards the first position.

107. An infusion pump comprising:
a first sledge operable by a loading actuator, the first sledge configured to linearly displace a syringe plunger;
a second sledge that is operatively coupled to the first sledge; and
a lock engageable with the second sledge to retain the second sledge in a locked configuration, the lock configured to permit the second sledge to be driven by energy stored within an energy storing member to linearly displace the first sledge in a dispensing direction in an unlocked configuration.

108. The infusion pump of claim 107, further comprising a port in removable fluid communication with a syringe barrel, the syringe barrel associated with the syringe plunger, the port configured to removably couple to a fluid container wherein operation of the loading actuator urges fluid flow from the fluid container to the syringe barrel when the lock is in the unlocked configuration.

109. The infusion pump of claim 108, wherein the port is configured to removably couple to an infusion set.

110. The infusion pump of claim 107, further comprising a rate actuator that is operatively coupled to the energy storing member, the rate actuator configured to mechanically adjust energy stored within the energy storing member.

111. The infusion pump of claim 107, wherein energy stored within the energy storing member is configured to linearly displace the second sledge at a substantially constant rate.

112. The infusion pump of claim 107, further comprising:
a first gear that is operatively coupled to the lock; and
a second gear that is operatively coupled to the first sledge, the second gear configured to engage with the first gear, wherein a gear ratio between the second gear and the first gear is greater than 1:1.

113. The infusion pump of claim 107, wherein the lock is configured to releasably de-couple the first sledge and the loading actuator when the lock is in the locked configuration.

114. The infusion pump of claim 107, wherein the second sledge is configured to apply a substantially constant force to the first sledge to displace the first sledge when the lock is in the unlocked configuration.

115. The infusion pump of claim 107, wherein the lock comprises a ratchet.

116. The infusion pump of claim 107, wherein the lock comprises a pad and brake, the brake configured to frictionally engage with the pad.

117. The infusion pump of claim 107, wherein the infusion pump is non-electric.

118. The infusion pump of claim 107, wherein the second sledge is configured to linearly displace the first sledge in only the dispensing direction when the lock is in the unlocked configuration.

119. A method of operating an infusion pump, the method comprising:
operating a loading actuator to linearly displace a first sledge, the first sledge configured to linearly displace a syringe plunger;
retaining a second sledge in a locked configuration with a lock engageable with the second sledge, wherein the second sledge is operatively coupled to the first sledge; and
unlocking the lock to permit the second sledge to be driven by energy stored within an energy storing member to linearly displace the first sledge in a dispensing direction in an unlocked configuration.

120. The method of claim 119, wherein operating the loading actuator comprises urging fluid flow from a fluid container that is removably coupled to a port of the infusion pump.

121. The method of claim 120, further comprising removably coupling the port to an infusion set, wherein the port is in removable fluid communication with a syringe barrel associated with the syringe plunger.

122. The method of claim 120, further comprising mechanically adjusting energy stored within an energy storing member.

123. The method of claim 122, further comprising linearly displacing the second sledge at a substantially constant rate with the energy stored within the energy storing member.

124. The method of claim 119, further comprising de-coupling the first sledge and the loading actuator when the lock is in the locked configuration.

125. The method of claim 119, further comprising applying a substantially constant force to the first sledge with the second sledge to displace the first sledge when the lock is in the unlocked configuration.

126. The method of claim 125, further comprising converting a variable output force of the energy storing member into the substantially constant force applied to the first sledge by a substantially inflexible connector wire that is operatively coupled between the second sledge and the energy storing member.

127. The method of claim 119, further comprising linearly displacing the first sledge with the second sledge in only the dispensing direction when the lock is in the unlocked configuration.

128. An infusion pump comprising:
a holder configured to removably couple to a syringe and to push and pull at least a portion of the syringe;
a loading actuator that is operatively coupled to the holder, the loading actuator configured to displace the holder; and
a port in removable fluid communication with the syringe, the port configured to removably couple to a fluid container,
wherein operation of the loading actuator urges fluid flow from the fluid container to the syringe.

129. The infusion pump of claim 128, further comprising a bubble trap that is in removable fluid communication with the port, wherein the bubble trap is configured to reduce air bubbles.

130. The infusion pump of claim 128, further comprising a lock moveable between a locked configuration and an unlocked configuration, the lock engageable with the holder to block fluid flow out of the syringe when the lock is in the locked configuration, the lock configured to permit fluid flow out of the syringe by allowing the holder to be driven by energy stored within an energy storing member to linearly displace the holder in a dispensing direction when the lock is in the unlocked configuration.

131. The infusion pump of claim 130, further comprising a rate actuator that is operatively coupled to the energy storing member, the rate actuator configured to mechanically adjust energy stored within the energy storing member.

132. The infusion pump of claim 130, wherein energy stored within the energy storing member is configured to linearly displace the holder at a substantially constant rate.

133. The infusion pump of claim 130, wherein the lock is configured to releasably de-couple the holder and the loading actuator when the lock is in the locked configuration.

134. The infusion pump of claim 128, wherein the loading actuator is rotatable.

135. A method of operating an infusion pump, the method comprising:
operating a loading actuator that is operatively coupled to a holder to displace the holder, the holder configured to removably couple to a syringe and to push and pull at least a portion of the syringe;
displacing the holder with the loading actuator; and
removably coupling a fluid container to a port, wherein the port is in removable fluid communication with the syringe,
wherein operation of the loading actuator urges fluid flow from the fluid container to the syringe.

136. The method of claim 135, further comprising:
blocking fluid flow out of the syringe with a lock engageable with the holder when the lock is in a locked configuration; and
permitting fluid flow out of the syringe by allowing the holder to be driven by energy stored within an energy storing member to linearly displace the holder in a dispensing direction when the lock is in an unlocked configuration.

137. The method of claim 135, further comprising removably coupling the port to an infusion set.

138. The method of claim 135, further comprising mechanically adjusting energy stored within an energy storing member.

139. The method of claim 136, further comprising de-coupling the holder and the loading actuator when the lock is in the locked configuration.

140. The method of claim 136, wherein permitting fluid flow out of the syringe comprises permitting fluid flow out of the syringe at a substantially constant rate.

141. An infusion pump comprising:
a holder configured to removably couple to a syringe plunger and to push and pull the syringe plunger;
a rate actuator that is operatively coupled to the holder;
an energy storing member that is operatively coupled between the rate actuator and the holder, wherein operation of the rate actuator mechanically adjusts energy stored within the energy storing member; and
a lock engageable with the holder to permit force transfer between the energy storing member and the holder to displace the holder when the lock is in an unlocked configuration.

142. The infusion pump of claim 141, wherein the energy storing member is configured to provide a variable output force.

143. The infusion pump of claim 142, further comprising:
a drive arm configured to apply a substantially constant force to the holder; and
a substantially inflexible connector wire that is operatively coupled between the drive arm and the energy storing member, the substantially inflexible connector wire configured to convert the variable output force of the energy storing member into the substantially constant force.

144. The infusion pump of claim 143, wherein the drive arm is configured to linearly displace the holder in only a dispensing direction when the lock is in the unlocked configuration.

145. The infusion pump of claim 141, further comprising a port in removable fluid communication with a syringe barrel, the syringe barrel associated with the syringe plunger, the port configured to removably couple to a fluid container wherein force transfer between the energy storing member and the holder urges fluid flow from the fluid container to the syringe barrel when the lock is in the unlocked configuration.

146. The infusion pump of claim 145, wherein the port is configured to removably couple to an infusion set.

147. The infusion pump of claim 141, further comprising:
a first gear that is operatively coupled to the lock; and
a second gear that is operatively coupled to the holder, the second gear configured to engage with the first gear, wherein a gear ratio between the second gear and the first gear is greater than 1:1.

148. The infusion pump of claim 141, further comprising a loading actuator that is operatively coupled to the holder, the loading actuator configured to displace the holder, wherein the lock is configured to releasably de-couple the holder and the loading actuator when the lock is in a locked configuration.

149. The infusion pump of claim 141, wherein the lock comprises a ratchet.

150. The infusion pump of claim 141, wherein the lock comprises a pad and brake, the brake configured to frictionally engage with the pad.

151. The infusion pump of claim 141, wherein the infusion pump is non-electric.

152. A method of operating an infusion pump, the method comprising:
operating a rate actuator to mechanically adjust energy stored within an energy storing member, wherein the energy storing member is operatively coupled between the rate actuator and a holder, the holder configured to removably couple to a syringe plunger and to push and pull the syringe plunger; and
permitting force transfer between the energy storing member and the holder by arranging a lock in an unlocked configuration, wherein the lock is engageable with the holder.

153. The method of claim 152, further comprising:
applying a substantially constant force to the holder with a drive arm; and
converting a variable output force of the energy storing member into a substantially constant force applied to the holder by a substantially inflexible connector wire that is operatively coupled between the drive arm and the energy storing member.

154. The method of claim 153, further comprising linearly displacing the holder with the drive arm in only a dispensing direction when the lock is in the unlocked configuration.

155. The method of claim 152, further comprising operating a loading actuator to urge fluid flow from a fluid container in removable fluid communication to a port to a syringe barrel when the lock is in the unlocked configuration.

156. The method of claim 155, further comprising de-coupling the holder and the loading actuator when the lock is in the locked configuration.

157. An infusion pump comprising:
a locking actuator configured to permit fluid flow out of a syringe in an unlocked configuration, the locking actuator configured to block fluid flow out of the syringe in a locked configuration;
a ratchet wheel configured to engage with a portion of the locking actuator in the locked configuration of the locking actuator, the ratchet wheel being rotationally coupled to a first gear; and
a second gear configured to engage with the first gear, the second gear being operatively coupled to the syringe, wherein a gear ratio between the second gear and the first gear is greater than 1:1.

158. The infusion pump of claim 157, further comprising a loading actuator that is operatively coupled to the syringe, the loading actuator configured to linearly displace the syringe.

159. The infusion pump of claim 158, wherein the locking actuator is configured to releasably de-couple the syringe and the loading actuator when the locking actuator is in the locked configuration.

160. The infusion pump of claim 157, wherein the portion of the locking actuator is a pawl.

161. The infusion pump of claim 157, wherein the gear ratio between the second gear and the first gear is greater than 3:1.

162. A method of operating an infusion pump, the method comprising:
permitting fluid flow out of a syringe by arranging a locking actuator in an unlocked configuration; and
blocking fluid flow out of the syringe by arranging the locking actuator in a locked configuration to engage a ratchet wheel with a portion of the locking actuator, wherein the ratchet wheel is rotationally coupled to a first gear, wherein the first gear is configured to engage with a second gear that is operatively coupled to the syringe, and wherein a gear ratio between the second gear and the first gear is greater than 1:1.

163. The method of claim 162, further comprising linearly displacing the syringe with a loading actuator.

164. The method of claim 163, wherein blocking fluid flow out of the syringe further comprises releasably de-coupling the syringe and the loading actuator when the locking actuator is in the locked configuration.

165. The method of claim 162, wherein the gear ratio between the second gear and the first gear is greater than 3:1.

166. The method of claim 163, further comprising operating the loading actuator to urge fluid flow from a fluid container that is removably coupled to a port of the infusion pump.

167. An infusion pump comprising:
a holder configured to removably couple to a syringe plunger and to push and pull the syringe plunger;
a loading actuator that is operatively coupled to the holder, wherein operation of the loading actuator causes the holder to push and pull the syringe plunger; and
a lock configured to permit fluid flow out of a syringe barrel associated with the syringe plunger in an unlocked configuration, the lock configured to block fluid flow out of the syringe barrel in a locked configuration, and the lock configured to releasably de-couple the holder and the loading actuator in the locked configuration.

168. The infusion pump of claim 167, wherein the lock comprises a clutch that is operatively coupled to the loading actuator, the clutch configured to de-couple the holder and the loading actuator.

169. The infusion pump of claim 167, wherein the lock is configured to permit fluid flow out of the syringe barrel by permitting the holder to be driven by energy stored within an energy storing member to linearly displace the holder when the lock is in the unlocked configuration.

170. The infusion pump of claim 167, further comprising a port in removable fluid communication with the syringe barrel, the port configured to removably couple to a fluid container wherein operation of the loading actuator urges fluid flow from the fluid container to the syringe barrel when the lock is in the unlocked configuration.

171. The infusion pump of claim 169, further comprising a rate actuator that is operatively coupled to the energy storing member, the rate actuator configured to mechanically adjust energy stored within the energy storing member.

172. The infusion pump of claim 169, wherein energy stored within the energy storing member is configured to linearly displace the holder at a substantially constant rate.

173. The infusion pump of claim 171, wherein the holder is configured to linearly displace the syringe plunger at a substantially constant speed, wherein the substantially constant speed is adjustable by the rate actuator.

174. The infusion pump of claim 167, wherein the lock comprises a ratchet.

175. The infusion pump of claim 167, wherein the lock comprises a pad and brake, the brake configured to frictionally engage with the pad.

176. A method of operating an infusion pump, the method comprising:
operating a loading actuator to linearly actuate a holder, the holder configured to removably couple to a syringe plunger, the holder configured to push and pull the syringe plunger;
permitting fluid flow out of a syringe barrel associated with the syringe plunger by arranging a lock in an unlocked configuration;
blocking fluid flow out of the syringe barrel by arranging the lock in a locked configuration; and
releasably de-coupling the holder and the loading actuator in the locked configuration.

177. The method of claim 176, further comprising releasably de-coupling the holder and the loading actuator with a clutch, wherein the clutch forms part of the lock.

178. The method of claim 176, wherein permitting fluid flow out of the syringe barrel comprises permitting the holder to be driven by energy stored within an energy storing member to linearly displace the holder when the lock is in the unlocked configuration.

179. The method of claim 176, wherein operating the loading actuator comprises urging fluid flow from a fluid container that is removably coupled to a port to the syringe barrel when the lock is in the unlocked configuration, the port in removable fluid communication with the syringe barrel.

180. The method of claim 178, further comprising mechanically adjusting energy stored within the energy storing member.

181. The method of claim 178, further comprising linearly displacing the holder at a substantially constant rate with the energy stored within the energy storing member.

182. The method of claim 181, further comprising adjusting the substantially constant rate.

183. An infusion pump comprising:
a holder configured to removably couple to a syringe plunger and to push and pull the syringe plunger; and
an energy storing assembly configured to apply a substantially constant force to the holder to displace the holder, the energy storing assembly comprising:
an energy storing member configured to provide a variable output force,
a drive arm configured to apply the substantially constant force to the holder, and
a linkage that is operatively coupled between the drive arm and the energy storing member, the linkage configured to convert the variable output force of the energy storing member into the substantially constant force.

184. The infusion pump of claim 183, wherein the linkage is a substantially inflexible connector wire.

185. The infusion pump of claim 183, further comprising a rate actuator that is operatively coupled to the energy storing assembly, the rate actuator configured to mechanically adjust a magnitude of the substantially constant force.

186. The infusion pump of claim 183, further comprising a lock that is operatively coupled to the holder, the lock engageable with the holder to permit force transfer between the energy storing member and the holder to displace the holder towards a dispensing direction when the lock is in an unlocked configuration.

187. The infusion pump of claim 183, further comprising a lock that is operatively coupled to the holder, the lock engageable with the holder to block force transfer between the energy storing member and the holder when the lock is in a locked configuration.
